(19) United States
(12) Patent Application Publication
(10) Pub. No.: US 2024/0382580 A1
(43) Pub. Date: Nov. 21, 2024

(54) MULTIVALENT VIRAL PARTICLES

(71) Applicant: CORNELL UNIVERSITY

(72) Inventor: Hector AGUILAR-CARRENO, Ithaca, NY (US)

(73) Assignee: CORNELL UNIVERSITY, Ithaca, NY (US)

(21) Appl. No.: 18/686,564

(22) PCT Filed: Aug. 26, 2022
(86) PCT No.: PCT/US2022/041703

§ 371 Date: Feb. 26, 2024

Related U.S. Application Data
(60) Provisional application No. 63/237,714, filed on Aug. 27, 2021.

(51) Int. Cl. A61K 39/205; A61K 39/00; A61K 39/12; A61K 39/145; A61K 39/155; A61K 39/215; A61K 39/275; C12N 7/00

(52) U.S. Cl. CPC A61K 39/205; A61K 39/12; A61K 39/145; A61K 39/155; A61K 39/215; A61K 39/275; C12N 7/00; A61K 2039/55555; A61K 2039/70; C12N 2710/24034; C12N 2760/12234; C12N 2760/14134; C12N 2760/16133; C12N 2760/18234; C12N 2760/18434; C12N 2760/20234; C12N 2770/20034

(57) ABSTRACT

The present disclosure is directed to Compositions comprising a population of multivalent viral particles are described herein. Also described herein are immunogenic compositions comprising a population of multivalent viral particles. Additionally, methods of making a multivalent immunogenic composition comprising a population of multivalent viral particles are described. Lastly, methods of using an immunogenic composition to elicit an immune response in a subject are described herein.

### CROSS REFERENCE TO RELATED APPLICATIONS

This application claims the benefit of U.S. Provisional Patent Application No. 63/237,714, filed Aug. 27, 2021, the contents of which is incorporated herein by reference in its entirety.

### STATEMENT REGARDING FEDERALLY SPONSORED RESEARCH OR DEVELOPMENT

This work was supported in part by Defense Advanced Research Project Agency (DARPA) administered through Cooperative Agreement D18AC00031-PREEMT. This study was supported by grants from NIH/NIAID R01 AI109022 and R21 AI142377. The government has certain rights in this invention.

### BACKGROUND

Nipah virus (NiV), Hendra virus (HeV) and Ebola virus (EBOV) are highly lethal zoonotic viruses requiring biosafety level 4 (BSL-4) containment. NiV and HeV cause encephalitis and respiratory disease in humans and susceptible animals with a mortality rate of 40-100%. Bishop K. A and Broder C. C, Lethal zoonotic paramyxoviruses. Washington DC: American Society of Microbiology, 2008; C. C. Broder et al., “A treatment for and vaccine against the deadly Hendra and Nipah viruses,” Antiviral Research, vol. 100, no. 1. 2013. doi: 10.1016/j.antiviral.2013.06.012; K. C. Ong and K. T. Wong, “Henipavirus encephalitis: Recent developments and advances,” Brain Pathology, vol. 25, no. 5, 2015, doi: 10.1111/bpa.12278; V. Sharma, et al., “Emerging trends of Nipah virus: A review,” Reviews in Medical Virology, vol. 29, no. 1. 2019. doi: 10.1002/rmv.2010; B. S. P. Ang, et al., “Nipah virus infection,” Journal of Clinical Microbiology, vol. 56, no. 6, 2018, doi: 10.1128/JCM.01875-17. EBOV causes a multisystem disease in humans and nonhuman primates, with a mortality rate up to 90% in humans. E. I. Ohimain, “Recent advances in the development of vaccines for Ebola virus disease,” Virus Research, vol. 211. 2016. doi: 10.1016/j.virusres.2015.10.021. Diseases caused by NiV, HeV, and EBOV have huge impacts on human and animal health. The first Asian NiV outbreak in 1998-1999 alone led to the slaughter of more than a million pigs and >100 human deaths. K. B. Chua, “Nipah virus outbreak in Malaysia,” Journal of Clinical Virology, vol. 26, no. 3, 2003, doi: 10.1016/S1386-6532(02)00268-8. The 2014 EBOV outbreak in West Africa caused >11,000 human deaths from >28,000 cases which led the World Health Organization to declare the outbreak a Public Health Emergency of International Concern (PHEIC). M. D. van Kerkhove, et al., “A review of epidemiological parameters from Ebola outbreaks to inform early public health decision-making,” Scientific Data, vol. 2. 2015. doi: 10.1038/sdata.2015.19; M. D. Ahmad, et al., “Optimal control analysis of Ebola disease with control strategies of quarantine and vaccination,” Infectious Diseases of Poverty, vol. 5, no. 1, 2016, doi: 10.1186/s40249-016-0161-6. Nipah virus has caused multiple outbreaks with high mortality rates averaging 75% in Asia. K. B. Chua, “Nipah virus outbreak in Malaysia,” Journal of Clinical Virology, vol. 26, no. 3, 2003, doi: 10.1016/S1386-6532(02)00268-8; N. Homaira et al., “Nipah virus outbreak with person-to-person transmission in a district of Bangladesh, 2007,” Epidemiology and Infection, vol. 138, no. 11, 2010, doi: 10.1017/S0950268810000695; G. Arunkumar et al., “Outbreak investigation of nipah virus disease in Kerala, India, 2018,” Journal of Infectious Diseases, vol. 219, no. 12, 2019, doi: 10.1093/infdis/jiy612; V. A. Narayan, “Nipah virus outbreak in India: is it a bat-man conflict?,” International Journal Of Community Medicine And Public Health, vol. 6, no. 4, 2019, doi: 10.18203/2394-6040.ijcmph20191430; R. Hussain, et al., “Nipah Virus Outbreak in India,” Indian Journal of Pharmacy Practice, vol. 12, no. 3, 2019, doi: 10.5530/ijopp.12.3.34. HeV has caused several outbreaks in Australia and EBOV in Africa Bishop K. A and Broder C. C, Lethal zoonotic paramyxoviruses. Washington DC: American Society of Microbiology, 2008; E. I. Ohimain, “Recent advances in the development of vaccines for Ebola virus disease,” Virus Research, vol. 211. 2016. doi: 10.1016/j.virusres.2015.10.021; A. T. Peterson and A. M. Samy, “Geographic potential of disease caused by Ebola and Marburg viruses in Africa,” Acta Tropica, vol. 162, 2016, doi: 10.1016/j.actatropica.2016.06.012.

Much evidence supports the notion that the main reservoir host for all three viruses are fruit bats in the family Pteropodidae, such as those in the genera Pteropus and Eidolon. However, evidently henipaviruses and EBOV are spreading into newer territories. This is associated with the spreading distribution of fruit bats caused by deforestation, climate change, and human movement. O. Pernet et al., “Evidence for henipavirus spillover into human populations in Africa,” Nature Communications, vol. 5, 2014, doi: 10.1038/ncomms6342; C. M. Mbu'U et al., “Henipaviruses at the Interface between Bats, Livestock and Human Population in Africa,” Vector-Borne and Zoonotic Diseases, vol. 19, no. 7. 2019. doi: 10.1089/vbz.2018.2365. A number of countries have reported henipavirus outbreaks or are at risk based on serological or molecular detection in Pteropus bats and the home range of Pteropus bats being widely spread (CDC 2014). CDC, “Henipa virus distribution maps,” published Mar. 20, 2014 on the website of CDC. For instance, antibodies against henipaviruses have been detected in Ghanaian bats in West Africa. O. Pernet et al., “Evidence for henipavirus spillover into human populations in Africa,” Nature Communications, vol. 5, 2014, doi: 10.1038/ncomms6342; C. M. Mbu'U et al., “Henipaviruses at the Interface between Bats, Livestock and Human Population in Africa,” Vector-Borne and Zoonotic Diseases, vol. 19, no. 7. 2019. doi: 10.1089/vbz.2018.2365. The distribution of these reservoir hosts helps predict the potential origin of these viral diseases and calls for a common vaccine platform for combating them, providing the motive for this study.

Henipavirus genomes consist of six genes N, P, M, F, G and L which encode nine proteins. R. Adiga, “EMERGENCE OF NIPAH VIRUS: A REVIEW,” Journal of Biomedical and Pharmaceutical Research, vol. 8, no. 1, 2019, doi: 10.32553/jbpr.v8i1.576; L. F. Wang et al., “Molecular biology of Hendra and Nipah viruses,” Microbes and Infection, vol. 3, no. 4. 2001. doi: 10.1016/S1286-4579(01)01381-8. The receptor binding glycoprotein (G) and fusion glycoprotein (F) facilitate viral attachment and entry into cells, respectively and are key antigens in generating neutralizing antibody responses G. P. Johnston et al., “Cytoplasmic Motifs in the Nipah Virus Fusion Protein Modulate Virus Particle Assembly and Egress,” Journal of Virology, vol. 91, no. 10, 2017, doi: 10.1128/jvi.02150-16; A. Tamin, et al., “Functional properties of the fusion and attachment glycoproteins of Nipah virus,” Virology, vol. 296, no. 1, 2002, doi: 10.1006/viro.2002.1418. Ebolaviruses encode nine proteins NP, GP, soluble GP (sGP), small soluble GP (ssGP), L and four structural proteins termed VP24, VP30, VP35, and VP40 A. Sanchez and P. E. Rollin, Virus Research, vol. 113, no. 1, 2005, doi: 10.1016/j.virusres.2005.03.028. The EBOV glycoprotein (GP) is the only virally expressed protein on the virion surface and is critical for attachment to host cells and execution of membrane fusion during viral entry A. Sanchez, et al., Virus Research, vol. 29, no. 3, 1993, doi: 10.1016/0168-1702(93)90063-S. The EBOV GP should be a vital component of vaccines, as it is targeted by neutralizing antibodies and inhibitors of attachment and fusion. GP, sGP, and ssGP are produced from the GP gene by alternative RNA editing. A. Sanchez and P. E. Rollin, Virus Research, vol. 113, no. 1, 2005, doi: 10.1016/j.virusres.2005.03.028.

There are currently no NiV and HeV vaccines licensed for human use; however, a recombinant replication incompetent VSV vaccine has been produced and licensed in the United States for Ebola virus. C. R. Piszczatoski and J. G. Gums, Journal of Pharmacy Technology, vol. 36, no. 6. 2020. doi: 10.1177/8755122520950692; E. Ollmann Saphire, Cell, vol. 181, no. 1, 2020, doi: 10.1016/j.cell.2020.03.011; M. D. van Kerkhove, et al., Scientific Data, vol. 2. 2015. doi: 10.1038/sdata.2015.19. Additionally, a soluble G glycoprotein vaccine for HeV is available for animal use in Australia. C. C. Broder et al., Antiviral Research, vol. 100, no. 1. 2013. doi: 10.1016/j.antiviral.2013.06.012. Monovalent Nipah virus-like particles (VLPs) have been produced, incorporating attachment and fusion glycoproteins. J. A. Pallister et al., Virology Journal, vol. 10, 2013, doi: 10.1186/1743-422X-10-237. VLPs are safe as they are replication incompetent, present the viral glycoproteins in their native conformation and are generally good immunogens, and their use also allows Differentiating Infected from Vaccinated Animals (DIVA) which is critical in areas where animal vaccination may be implemented. G. T. Jennings and M. F. Bachmann, Annual Review of Pharmacology and Toxicology, vol. 49. 2009. doi: 10.1146/annurev-pharmtox-061008-103129. The VSV platform has been used for production of many vaccines which are either replication competent or incompetent. M. A. Whitt, et al., Methods in Molecular Biology, vol. 1403, 2016. doi: 10.1007/978-1-4939-3387-7_16.

Experimental vaccines for Nipah (NiV), Hendra (HeV), and Ebola (EBOV) viruses have focused on targeting individual viruses, although their geographical and bat reservoir host overlaps warrant creation of multivalent vaccines. The current global pandemic has made it abundantly clear that we need vaccines that are ready to prevent future emerging pandemics. The emerging paramyxoviruses, NiV and HeV, and the filovirus, EBOV, are Risk Group 4 pathogens that cause encephalitis, respiratory disease, and/or multisystemic disease that result in 40-90% mortality rates in humans. Despite exploration of several promising vaccine platforms, only the recently licensed Ervebo vaccine, for prevention of Ebola virus disease, is licensed for use in humans. The geographical and bat host overlaps among these viruses invite the creation of multivalent vaccines that target all these important and potentially pandemic viruses.

Studies on vaccines for NiV, HeV and EBOV have focused on individual viruses. However, the wide distribution of Pteropus fruit bats that carry these viruses, added to their human-to-human transmission, invites scenarios of rapid and widespread global disease.

Rift valley fever virus is zoonotic. It is a tri-segmented negative strand virus in the family bunyaviridae causing disease in sheep, goats, cattle and humans in Africa and the Middle East. K. L. Mansfield, et al. Vaccine 33 (2015) 5520-5531. Serological tests show the virus can infect camels, buffalo and warthogs. Id. The disease in sheep cause neonatal deaths approaching 90% and 10-30% mortality in adults. It is also characterized by an abortion storm in almost 100% of affected ewes. In humans, the disease cause 1-5% mortalities but a recent outbreak in Kenya and Sudan resulted in human fatality rate approaching 20%. Id. RVFV is also considered a potential agro- and bio-terror agent. It therefore continues to pose a threat to livestock and public health.

Peste des petits ruminants virus (PPRV) is a Paramyxovirus in the genus Morbillivirus with a single negative strand RNA genome. It causes disease primarily in sheep and goats with a mortality of 70-80%. S. Parida, et al., Veterinary Microbiology 181 (2015) 90-106. Whereas disease due to PPRV has for a period been confined to Africa and the Middle East, disease outbreaks have recently occurred in China. Id. In the early 2000s, the Food and Agriculture Organization (FAO) singled out PPR as one of the principal diseases when considering policies pertaining to poverty alleviation (African Union International Bureau for Animal Resources, 2012) and International Office of Epizootics (OIE) targets PPRV for eradication after Rinderpest. Id. In the last phase of a disease eradication program, it is important to have a vaccine that allows differentiating infected and vaccinated animals (DIVA). The current live attenuated PPRV vaccine does not exhibit this character. The PPRV component of the proposed multivalent RVFV-PPRV-GTPV vaccine will address this gap while reducing livestock decimation due to PPRV disease outbreaks.

Goat pox virus (GTPV) is a double stranded DNA virus in the family Poxviridae and the genus Capripox viruses. It is closely related to other two Capripox viruses; Sheep pox virus (SPV) that affect sheep and Lumpy skin disease virus (LSDV) that affect cattle. E. S. M. Tuppurainen, et al., Transboundary and Emerging Diseases (2015), doi:10.1111/tbed.12444. The Capripox viruses have distributions in Africa and Asia causing morbidity in flocks of 70-90% and mortalities of up to 50% in adults. Mortalities in young flocks are up to 100%. Id. Diseases caused by Capripox viruses are OIE listed notifiable diseases. Among the Capripox viruses, the P32 attachment protein is conserved and has up to 97% homology. E. R. Tulman, et al., J. Virol. 76, (2002) 6054-6061. Indeed, there is serological cross-reaction and cross-protection among these viruses. G. Gari, et al., Vaccine 33, (2015) 3256-3261. It is envisaged that the RVFV-PPRV-GTPV vaccine may protect against SPV and LSDV. This will have huge economic benefits in areas where these diseases occur together.

A striking feature for the RVFV, PPRV and GTPV is the overlap in their distribution and the chronology of the disease patterns. During wet weather, RVFV is more prevalent. As the rains subside and fodder for animals get scarce, there is mixing of animals in the pastoral grazing fields and animal watering points especially in arid and semi-arid lands in Africa. These conditions promote transmission of PPRV and Capripox viruses.

The current COVID-19 pandemic has had huge devastating impact on global health and economy. S. A. Sarkodie and P. A. Owusu, Environment, Development and Sustainability, 2020, doi: 10.1007/s10668-020-00801-2. The mechanisms of COVID-19 transmission are also shared among many other viruses. H. A. Rothan and S. N. Byrareddy, Journal of Autoimmunity, vol. 109. 2020. doi: 10.1016/j.jaut.2020.102433; M. A. Shereen, et al., Journal of Advanced Research, vol. 24. 2020. doi: 10.1016/j.jare.2020.03.005. This pandemic emphasizes the need for the global community to be prepared for virus disease outbreaks, such as by the preemptive development of prophylactic vaccines. Some of the deadliest viruses of concern are the subject of this study.

Influenza virus is a negative-sense RNA virus belonging to the family Orthomyxoviridae. Flu is the 7th leading cause of death in the US according to the Center for Disease Control. Influenza A affects both humans and animals. The main challenge with influenza A virus is antigenic drift which results in many variants. Current flu vaccines are whole inactivated vaccines which protects against the influenza A (subtype: H1N1, H3N2), and influenza B strain. Sook-San Wong, Richard J. Webby. ASM Journal, 2013. Influenza whole inactivated vaccines mainly induce antibodies targeting hemagglutinin, a surface glycoprotein used for viral entry.

### BRIEF SUMMARY OF THE DISCLOSURE

The present disclosure is directed to a population of multivalent viral particles, an immunogenic composition comprising a population of particles, methods of making a multivalent immunogenic composition, and methods of using an immunogenic composition to elicit an immune response in a subject.

In a first aspect, the present disclosure is directed to a composition comprising a population of particles that are replication-incompetent virions or virus-like particles, wherein the particles each comprise (i) a matrix glycoprotein base, wherein the matrix glycoprotein is from a first virus species, and (ii) surface glycoproteins from at least two virus species. In some embodiments, the composition comprising a population of particles that are replication-incompetent virions or virus-like particles comprising at least two virus species include a virus species different from the first virus species. In some embodiments, the at least two virus species include virus species from two virus families. In some embodiments, the virus families are selected from filoviruses, rhabdoviruses, coronaviruses, arenaviruses, orthomyxoviruses, retroviruses, flaviviruses, togaviruses, alphaviruses, and paramyxoviruses. In some embodiments, the first virus species is selected from Nipah (NiV), Hendra (HeV), Ebola (EBOV), Rift valley fever (RVFV), peste des petits ruminants (PPRV), goat pox (GTPV), influenza viral species, and beta-coronaviral species. In some embodiments, the beta-coronaviral species are SARS-CoV-1, SARS-CoV-2, MERS-CoV, and their variants. In some embodiments, the surface glycoproteins are selected from NiV-F, NiV-G, HeV-F, HeV-G, and EBOV-GP. In some embodiments, the surface glycoproteins are selected from SARS-CoV-1 spike protein, SARS-CoV-2 spike protein, and MERS-CoV spike protein. In some embodiments, the surface glycoproteins are selected from RVFV GnGc, PPRV F and H, and GTPV P32. In some embodiments, the surface glycoproteins are selected from SARS-CoV-2 spike protein and influenza neuraminidase (NA).

Some aspects of the disclosure are directed to a composition comprising a population of particles that are replication-incompetent virions or virus-like particles, wherein the particles each comprise (i) a matrix glycoprotein base, wherein the matrix glycoprotein is from a first virus species, and (ii) surface glycoproteins from at least two virus species and the population of particles is a replication-incompetent virion. In some embodiments, the virion is a pseudotyped vesicular stomatitis virus (VSV). In some embodiments, the pseudotyped VSV is a recombinant VSV wherein the native envelope G protein is removed. In some embodiments, the recombinant VSV native G protein is replaced by a reporter protein. In some embodiments, the virion comprises surface glycoproteins from two virus species. In some embodiments, the virion comprises surface glycoproteins from three virus species. In some embodiments, the virion comprises surface glycoproteins from virus species of at least two virus families. In some embodiments, the surface glycoproteins are from virus families selected from filoviruses, rhabdoviruses, coronaviruses, arenaviruses, orthomyxoviruses, retroviruses, flaviviruses, togaviruses, alphaviruses, and paramyxoviruses. In some embodiments, the surface glycoproteins are from viruses selected from Nipah (NiV), Hendra (HeV), Ebola (EBOV), Rift valley fever (RVFV), peste des petits ruminants (PPRV), goat pox (GTPV), influenza viral species, and beta-coronaviral species. In some embodiments, the beta-coronaviral species are SARS-CoV-1, SARS-CoV-2, MERS-CoV, and their variants. In some embodiments, the surface glycoproteins are selected from NiV-F, NiV-G, HeV-F, HeV-G, and EBOV-GP. In some embodiments, the surface glycoproteins are selected from SARS-CoV-1 spike protein, SARS-CoV-2 spike protein, and MERS-CoV spike protein. In some embodiments, the surface glycoproteins are selected from RVFV GnGc, PPRV F and H, and GTPV P32. In some embodiments, the surface glycoproteins are selected from SARS-CoV-2 spike protein and influenza neuraminidase (NA).

Some aspects of the present disclosure are directed to a composition comprising a population of particles that are replication-incompetent virions or virus-like particles, wherein the particles each comprise (i) a matrix glycoprotein base, wherein the matrix glycoprotein is from a first virus species, and (ii) surface glycoproteins from at least two virus species and the population of particles comprises a virus-like particle (VLP). In some embodiments, the VLP comprises a matrix protein selected from a virus in one of the following families: paramyxovirus, filovirus, flavivirus, orthomyxovirus, arenavirus, retrovirus, coronavirus, rhabdoviruses, togavirus, and alphavirus. In some embodiments, the matrix protein is from a virus in the paramyxovirus family. In some embodiments, the matrix protein is a Nipah virus matrix protein. In some embodiments, the matrix protein is a Hendra virus matrix protein. In some embodiments, the VLP comprises surface glycoproteins from two virus species. In some embodiments, the VLP comprises surface glycoproteins from three virus species. In some embodiments, the virion comprises surface glycoproteins from virus species of at least two virus families. In some embodiments, the surface glycoproteins are from viruses selected from the virus families of filoviruses, rhabdoviruses, coronaviruses, arenaviruses, orthomyxoviruses, retroviruses, flaviviruses, togaviruses, alphaviruses, and paramyxoviruses. In some embodiments, the surface glycoproteins are from viruses selected from two or more of the following Nipah (NiV), Hendra (HeV), Ebola (EBOV), Rift valley fever (RVFV), peste des petits ruminants (PPRV), goat pox (GTPV), influenza viral species, and beta-coronaviral species. In some embodiments, the beta-coronaviral species are SARS-CoV-1, SARS-CoV-2, MERS-CoV, and their variants. In some embodiments, the surface glycoproteins are selected from NiV-F, NiV-G, HeV-F, HeV-G, and EBOV-GP. In some embodiments, the surface glycoproteins are selected from SARS-CoV-1 spike protein, SARS-CoV-2 spike protein, and MERS-CoV spike protein. In some embodiments, the surface glycoproteins are selected from RVFV GnGc, PPRV F and H, and GTPV P32. In some embodiments, the surface glycoproteins are selected from SARS-CoV-2 spike protein and influenza neuraminidase (NA).

Some aspects of the current disclosure are directed to an immunogenic composition comprising a population of particles that are replication-incompetent virions or virus-like particles, wherein the particles each comprise (i) a matrix glycoprotein base, wherein the matrix glycoprotein is from a first virus species, and (ii) surface glycoproteins from at least two virus species and a pharmaceutically acceptable carrier. In some embodiments, the immunogenic composition further comprises a carbohydrate. In some embodiments, the carbohydrate is at a range of concentration between about 1 wt % and 10 wt %. In some embodiments, the carbohydrate is at a concentration of 5 wt %. In some embodiments, the carbohydrate is sucrose or trehalose. In some embodiments, the carbohydrate preserves the thermostability of the composition. In some embodiments of the composition is lyophilized.

Some aspects of the disclosure are directed to a method of eliciting an antibody response in a subject, the method comprising administering an immunogenic composition described herein to the subject in an amount sufficient to elicit an antibody response. In some embodiments, the subject is a mammal. In some embodiments, the subject is a mouse. In some embodiments, the subject is a human.

Some aspects of the disclosure are directed to a method of making a multivalent immunogenic composition, the method comprising:
providing a host cell comprising (i) a modified genomic DNA of a first virus species comprising a nucleic acid encoding a matrix glycoprotein, wherein the modification creates a replication incompetent virus, comprises a deletion of a gene encoding a surface glycoprotein; and (ii) nucleic acids encoding surface glycoproteins from at least 2 virus species;
infecting the host cell with a helper virus of the first virus species to produce a population of particles comprising replication incompetent virions comprising the matrix protein of the first virus species and the surface glycoproteins from at least 2 virus species; and isolating the population of particles.

In some embodiments, the helper virus is a Vesicular stomatitis virus (VSV).

Some aspects of the present disclosure are directed to a method of making a multivalent immunogenic composition, the method comprising:
transfecting a host cell with a nucleic acid encoding a matrix protein of one virus species and nucleic acids encoding surface glycoproteins of at least two virus species;
expressing viral proteins in the host cell to allow formation of particles comprising surface glycoproteins of at least two virus species;
purifying the particles; and formulating the multivalent immunogenic composition comprising the purified particles.

In some embodiments, the host cell is selected from bacteria, mammalian cells, insect cells, yeast, and plant cells. In some embodiments, the matrix protein is selected from a virus in one of the following families: paramyxovirus, filovirus, flavivirus, orthomyxovirus, arenavirus, retrovirus, coronavirus, matonavirus, togavirus, and alphavirus. In some embodiments, the matrix protein is from a virus in the paramyxovirus family. In some embodiments, the matrix protein is a Nipah virus matrix protein. In some embodiments, the matrix protein is a Hendra virus matrix protein. In some embodiments, the genes for viral surface glycoproteins of interest are selected from fusion proteins (F), receptor binding glycoproteins (G), glycoprotein (GP), spike proteins (S), Neuraminidase (NA), amino-terminus glycoprotein (Gn), carboxyl-terminus glycoprotein (Gc), hemagglutinin (H), and P32 protein. In some embodiments, the genes for viral surface glycoproteins of interest are selected from fusion proteins (F), receptor binding glycoproteins (G), and glycoprotein (GP). In some embodiments, the genes for viral surface glycoproteins of interest are selected from spike proteins (S) and neuraminidase (NA). In some embodiments, the genes for viral surface glycoproteins of interest are selected from amino-terminus glycoprotein (Gn), carboxyl-terminus glycoprotein (Gc), hemagglutinin (H), and P32 protein. In some embodiments, the genes for viral surface glycoproteins of interest are multiple spike proteins.

### BRIEF DESCRIPTION OF THE DRAWINGS

The patent or application file contains at least one drawing executed in color. Copies of this paper or patent application publication with color drawing(s) will be provided by the Office upon request and payment of the necessary fee.

FIG. 1A-F. Optimized production of NiV-HeV-EBOV VLPs and their protein detection using multiple techniques. A. Different amounts of DNA (μg) were used to transfect HEK 293T cells to determine the closest range for each target protein for optimized production detected by Western Blot analysis (WBA). B. Shows the best VLP production DNA ratio detected by WBA. C. WBA detection of proteins in cell lysate of cells transfected to produce VLPs in B. D. The cell surface expression (CSE) of the target proteins analyzed by Flow Cytometry (FC). E. Detection of the target proteins on VLPs using Flow Virometry (FV). F. A micrograph of the produced VLPs viewed using a FEI T20 electron microscope.

FIG. 2. A diagram showing transfection of HEK 293T cells using plasmids carrying DNA encoding for the immunogenic proteins of interest, followed by infection using VSV-deltaG. The subsequent pseudotyped VSV virion preparation carries all five distinct viral glycoproteins and can be used as a multivalent vaccine.

FIG. 3A-H. Optimized production of Pseudotyped VSV incorporating NiV-HeV-EBOV glycoproteins. A. Different amounts of DNA (μg) were used to give the closest range for each target protein for optimized production detected by WBA. B. WBA detection of proteins in cell lysate of cells transfected to produce the pseudotyped VSV particles. C. Shows the best DNA ratio for production of pseudotyped VSV detected by WBA. D. The cell surface expression (CSE) of the target proteins analyzed by FC. E. Detection of the target proteins on pseudotyped VSV using FV. F. A micrograph of the produced pseudotyped VSV particles viewed using a FEI T20 electron microscope. G. Flow cytometry data showing an example of the presence of double-positive viral populations (red) as compared to the negative control bald particles (blue). H. Percentage of viral populations double positive for various pairs of glycoproteins with extracellular tags. One representative experiment of three is shown.

FIG. 4A-F. Optimized production and co-expression of RVFV-PPRV-GTPV VLPs and their protein detection using Western blotting (WB) and electron microscopy (EM). A, B, C and D Target proteins were incorporated and detected by WB after transfecting HEK 293T cells with different amounts of expression plasmids for NiV M, RVFV GnGc, PPRV F and H, and GTPV P32. The protein constructs had the following DNA tags: NiV M-flag, PPRV F-flag, PPRV H-HA, RVFV GnGc-V5, and PPRV P32-Myc. VLPs budding into the supernatant were recovered by ultracentrifugation in a 20% sucrose gradient and stored at −80° C. in 5% sucrose/NTE. The VLPs were run on a Standard Western blot. The blots were stained with species anti-tag primary antibodies (mouse anti-flag, mouse anti-myc, rabbit anti-HA, mouse anti-V5) followed by anti-species fluorophore antibodies (anti-mouse 647 and anti-rabbit 488). The blots were imaged using BioRAD imager. E. is a WB detection of proteins in cell lysate of cells transfected to produce VLPs in D. F. An electron microscope micrograph of the produced VLPs. The VLPs were adsorbed on Formvar carbon coated copper grid by floating it on a drop of sample suspension for 15 minutes and fixed using 2% formaldehyde/2% glutaraldehyde solution in 0.1M cacodylate buffer. The grids were blotted and negatively stained with 1% aqueous uranyl acetate. They were viewed using a FEI T20 electron microscope.

FIG. 5A-D. Cell surface protein expression of RVFV GnGc, PPRV H, and GTPV P32 viral proteins in a VLP system (A, B) and a pseudotyped VSV system (C, D).

FIG. 6A-E. Optimized production and co-expression of RVFV-PPRV-GTPV pseudotyped VSV particles and their protein detection using Western blotting (WB) and electron microscopy (EM). A, B, and C. Target proteins were incorporated and detected by WB after transfecting HEK 293T cells with different amounts of expression plasmids for NiV M, RVFV GnGc, PPRV F and H, and GTPV P32. The cells were infected with VSVAG 12 hours after transfection. The protein constructs had the following DNA tags: NiV M-flag, PPRV F-flag, PPRV H-HA, RVFV GnGc-V5, and PPRV P32-Myc. Pseudotyped VSV budding into the supernatant were recovered by ultracentrifugation in a 20% sucrose gradient and stored at −80° C. in 5% sucrose/NTE. The VLPs were run on a Standard Western blot. The blots were stained with species anti-tag primary antibodies (mouse anti-flag, mouse anti-myc, rabbit anti-HA, mouse anti-V5) followed by anti-species fluorophore antibodies (anti-mouse 647 and anti-rabbit 488). The blots were imaged using BioRAD imager. D. is a WB detection of pseudotyped VSV proteins produced in a 15 cm plate. E. An electron microscope micrograph of the produced VLPs. The VLPs were adsorbed on Formvar carbon coated copper grid by floating it on a drop of sample suspension for 15 minutes and fixed using 2% formaldehyde/2% glutaraldehyde solution in 0.1M cacodylate buffer. The grids were blotted and negatively stained with 1% aqueous uranyl acetate. They were viewed using a FEI T20 electron microscope.

FIG. 7A-D. Carbohydrates preserve the thermostability of pseudotyped VSV particles. A. Vero cell entry capabilities for pseudotyped VSV incorporating monovalent and multivalent target proteins determined using Renilla Luciferase assay. The relative MOI of the pseudotyped VSV was determined based on luminescence produced from the Luc gene and read as relative light units (RLU). The pseudotyped VSV virions were collected 40 hours post transfection. The monovalent and multivalent pseudotyped VSV virions were diluted 1:100 to 1:1,000,000 and their entry capabilities determined to assess the optimal entry levels for the monovalent and multivalent pseudotyped VSV. B. Determination of Vero cell entry of non-lyophilized multivalent pseudotyped VSV exposed to 4° C., 25° C. and 37° C. for 42 days. C. Determination of sucrose and trehalose capability to preserve the multivalent pseudotyped VSV during lyophilization. D. Determination of viability for the 5% w/v trehalose preserved pseudotyped VSV. Viability was determined using the Renilla Luciferase assay

FIG. 8A-F. Multivalent VLPs and pseudotyped VSV incorporating NiV F/G, HeV F/G and EBOV GP elicited neutralizing antibodies in hamsters. A. Negative control hamsters were vaccinated with bald VLPs. Serum for the hamsters' terminal bleed was used to neutralize monovalent NiV F/G, HeV F/G, EBOV GP and the multivalent pseudotyped VSV particles and entry of the virus into the cells was analyzed by Renilla Luciferase assay. The mean neutralization read out for the monovalent and multivalent pseudotyped VSV was calculated, and graph plotted using Graphpad software. B. Monovalent and multivalent pseudotyped VSV were neutralized with different dilutions of sera from hamsters vaccinated with multivalent VLP vaccine. Virus neutralization was done in a similar manner as the negative controls. The average neuralization read outs were calculated and graph derived using Prism Graphpad software. C. Hamsters were vaccinated with the multivalent pseudotyped VSV. Sample processing was done as for the negative controls. The mean neutralization read out was calculated and graph derived using Prism graphpad software. D and E. Normalized graphs for sera from hamsters vaccinated with the multivalent VLPs and pseudotyped VSV. F. Forty microliters of VLP and pseudotyped VSV preparations were analyzed by Western blotting to compare incorporation of glycoproteins following observation that pseudotyped VSV was eliciting a relatively stronger immune response compared to VLPs.

FIG. 9. FIG. 9 indicates the vaccination schedule for all hamsters used in this study, the amounts of mock and test vaccines applied, the point of challenge, and the period that the challenge experiment lasted.

FIG. 10. Serum IgG ELISA binding quantification between groups that received mock, VLP, or VSV vaccinations. Hamster serum was diluted 1:10,000 and incubated for 30 minutes on plates provided by Abcam Hamster IgG ELISA Kit (ab200010). Wells were washed 3× using provided buffers and incubated with anti-hamster secondary antibodies for 30 minutes. Development solution was added for 10 minutes, then stopped using the provided stop solution. Quantification was done using a Tecan Spark recording the OD at 450 nm.

FIG. 11A-F. Determination of neutralizing antibodies against NiV/HeV and EBOV measured pre and post challenge. A to C. Neutralization curves for pseudotyped VSV vaccinated hamsters prior to challenge. A-C represent neutralizing antibodies measured using NiV, HeV, and EboV VSV using sera collected 1 week before challenge ˜5 weeks post last vaccination. D to F. FRNA50 detecting neutralizing Ab against live NiV/HeV and EBOV measured pre and post challenge. Average of all animals in each group error bars represent ±SD n=3.

FIG. 12A-D. Analysis of hamster survival, weight and temperatures during the course of challenge experiments. A. Percent survival of test vaccinated vs. mock-vaccinated hamsters, following challenge with live NiV, HeV, or maEBOV. Hamsters vaccinated with the test vaccine survived challenge with all the three virulent viruses except that one hamster died from a lymphoma (unrelated cause). Those vaccinated with mock VSV virions were euthanized at critical control points after signs of disease. B. Weight records for the test vs. mock vaccinated hamsters groups. Hamsters in the test vaccine group showed minimal variation on weight during the course of experiments while those in the mock-vaccinated groups registered significant weight loss prior to euthanasia. C. Body temperature records for the mock vs. test vaccinated groups. Hamsters test vaccinated showed minimal variation in body temperatures while the variation was marked for the mock groups. D. Clinical scores for the multivalent pseudotyped VSV vaccinated hamsters compared the mock treated group. Clinical scores were based on a 15-point scale that includes scoring appearance, respiratory signs, mobility, temperature, and neurological signs such as paralysis, seizures, and appearing moribund. Additive scores of 15 or greater required immediate consultation with the veterinarian to determine animal disposition. There was marked deviation of clinical scores between test and mock vaccinated groups. Solid lines represent test-vaccinated animals, while dashed lines represent mock vaccinated animals.

FIG. 13A-H. Multivalent VSV virion vaccination prevented the development of histologic lesions in hamsters challenged with Nipah, Hendra, or Ebola viruses. Tissues from representative mock vaccinated (A, C, E, G) or multivalent VSV virion vaccination (B, D, F, H) following challenge with either Nipah (A-D) or Ebola virus (E-H). A. Brain tissue from a representative mock vaccinated hamster with inflammatory perivascular cuffs (arrows). C. Lung tissue from a representative mock vaccinated hamster with intravascular fibrin thrombi (arrowhead), edema in alveoli (asterisk), and type II pneumocyte hyperplasia with atypia (arrow). E. Liver tissue from a from a representative mock vaccinated hamster with lobular hepatitis, hepatocyte necrosis (arrow), and intracytoplasmic viral inclusions (arrowhead). G. Lung tissue from a representative mock vaccinated hamster with intravascular fibrinocellular debris (arrows) and expansion of alveolar septa with inflammatory cells (arrowhead). Lesions caused by Hendra virus resembled those caused by Nipah virus. H&E, scale bar=50 μm.

FIG. 14A-C. Sera from hamsters vaccinated with multivalent pseudotyped VSV virions incorporating NiV F/G, HeV F/G and EBOV GP can bind CedV F and cross neutralize CedV F/G pseudotyped VSV virions. A. Diagram showing sequence identity among NiV, HeV and CedV G proteins. B. A comparison of the binding properties of serum to NiV F/G or CedV F/G. C. Comparison of entry properties of NiV and CedV pseudotyped VSV virions neutralized with serum from vaccinated hamster 6.2.

FIG. 15A-B. Determination of serum neutralization to RVFV and PPRV using sera from experimental mice. A. Graph showing neutralization of RVFV. C. Graph showing neutralization of PPRV.

FIG. 16A-B. Incorporation of CoV surface proteins onto VLPs. A. Preliminary incorporation of SARS-CoV-1, SARS-CoV-2, or MERS-CoV S glycoproteins into VLPs, all samples run on the same SDS-PAGE gel. B. Preliminary sub-optimized VLPs obtained using the indicated ratios of N:M:E:S expression plasmids, showing low to medium levels of incorporation of CoV proteins into VLPs. Note: all proteins were detected with different tags, so their incorporation levels cannot be quantitatively compared in this particular analysis.

FIG. 17A-B. Preliminary incorporation of CoV S glycoproteins into pseudotyped VSV particles, and viral entry levels into Vero TMPRRS2 cells. A. Viral entry levels of individual vs. bivalent SARS-CoV-2/MERS-CoV VSV-Renilla luciferase reporter pseudotyped virions. Relative light units (RLU) were measured using a Tecan plate reader. pCAGGS is the mock background control. B. Incorporation of individual or bivalent CoV spike glycoproteins onto pseudotyped virions. All samples were run on the same SDS-PAGE gel.

FIG. 18. Effect of SARS-CoV-2 inoculation dose on weight loss and survival in K18 hACE2 mice. 8-week old mice received an intranasal inoculation of the indicated doses from 3×10¹ to 3×10⁵ of the USA WA1 isolate of SARS-CoV-2, were monitored for weight loss and clinical signs for 14 days, and euthanized at end-point (>20% weight loss or severe clinical signs). Weight is expressed as percentage of individual weight on challenge day (day 0). Viral inoculation doses were 3×the indicated logarithmic # of plaque forming units (PFU)/mouse. K18 hACE2 mice were highly susceptible to SARS-CoV-2, with doses as low as 30 PFU/mouse inducing some clinical signs and some deaths. Higher inoculation doses were associated with higher probabilities of developing more severe clinical signs and shorter times from viral infection to euthanasia. Accompanying histology (H&E staining), immunohistochemistry (IHC), and viral loads (obtained by qPCR), were obtained for lung, brain, spleen, small and large intestines, and other tissues and were consistent with these results (data not shown).

FIG. 19A-D. Small pilot mouse experiment using a bivalent SARS-CoV-2/MERS-CoV S vaccine. A. Timetable of vaccination of K18 hACE 2 mice, with blood draws and euthanasia shown. Lung, brain, heart, intestines and liver were collected for further histological and viral load analysis (not yet completed for this particular experiment). Three male mice were used per group for this small pilot experiment. B. Normalized viral entry, relative light unit (RLU) of SARS CoV-2 or MERS-CoV VSV pseudotyped virions neutralized with mice sera collected at days 0, 14 and 21 post prime vaccination. C. Normalized average weight post challenge with 10′PFU SARS-CoV-2/mouse. D. Survival rate post challenge. Mock vaccine: VSV particles produced by transfection with pCAGGS vector plasmid only.

FIG. 20. Schematic showing influenza NA SARS-CoV2 S multivalent VSV vaccine production.

FIG. 21A-B. A. Graph representing the serum dilution and the percent SARS-CoV-2 inhibition. B. Graph representation of serum dilution and the percent influenza NA inhibition. The Bivalent VSV pseudotyped vaccine induces neutralizing antibodies against pseudotyped SARS-CoV-2 virus and H1N1-PR8 virus in mice.

### DETAILED DESCRIPTION

In one aspect, the current disclosure is directed to a population of multivalent viral particles. Some embodiments of the current disclosure are directed to a population of multivalent viral particles comprising multiple surface glycoproteins from more than one species of virus.

The term “multivalent” as used herein in the context of viral particles refers to the ability of the viral particles to induce immune response in a host to multiple viral species. In some embodiments, the viral particles are capable of inducing immune responses in a host to the multiple viral species from which the surface glycoproteins are derived or obtained. In some embodiments, the viral particles are capable of inducing immune responses in a host to additional viral species different from those that provide the surface glycoproteins. For example, as shown herein, cross-neutralization of a related henipavirus, Cedar virus (CedV) having moderate sequence similarities to NiV and HeV G and F was also neutralized when only NiV and HeV G and F glycoproteins were presented.

It is economically and logistically prudent to produce multivalent vaccines for diseases that have global and/or regional overlap in their occurrence. This disclosure demonstrates that immunogenic proteins from different virus species and families (including unrelated virus species and families) can be combined onto replication-incompetent VLP or pseudotyped VSV platforms to result in safe and effective vaccines. The findings disclosed herein are important for vaccine development, including vaccines for families of enveloped viruses, such as SARS-CoV-2 and related coronaviruses and other emerging viruses, as well as other established families of viruses.

In one exemplary embodiment of this disclosure, two or more surface glycoproteins from different viruses (i.e., different viral species) were incorporated onto VLPs or VSV pseudotyped virions for use as candidate multivalent VLP-based vaccines. Multivalent NiV-HeV-EBOV VLPs and pseudotyped VSV virions were produced, characterized, and tested using a Syrian hamster model of Henipavirus and Ebolavirus disease (which model is described in Hana M. Weingartl, et al., “Animal models of henipavirus infection: A review.” The Veterinary Journal, vol. 181, pp. 211-220, 2009). The effect of temperature on the VLP and pseudotyped VSV vaccines was addressed, and the vaccines were lyophilized in the presence of carbohydrates to improve their thermotolerance. Such improvement is necessary as these viruses cause disease in regions of the world that face challenges for maintaining vaccine cold-chain. The replication incompetent VSV multivalent vaccine showed superior incorporation of the glycoproteins and 100% efficacy and safety upon challenge with any of the NiV, HeV, and EBOV viruses.

As an example, one embodiment of the disclosure is directed to co-incorporation of multiple surface glycoproteins from NiV, HeV and EBOV onto VLPs or replication incompetent VSV virions. In some embodiments, the vaccines' thermotolerance is enhanced by way of carbohydrates. Such enhanced thermotolerance allows greater applicability in the disease-prevalent geographical regions that lack cold-chain infrastructure. In this example of the embodiments, the VSV-based virions incorporated the NiV, HeV, and EBOV surface glycoproteins more efficiently than the NiV VLP virions. Additionally, a Syrian hamster model of human disease for these three viruses showed the VSV multivalent vaccine elicited strong and protective neutralizing antibody responses against all three viruses, yielding 100% safety and 100% protective efficacy against challenge with any of the three deadly viruses NiV, HeV or EBOV.

### Viruses

Viruses are small obligate intracellular parasites, which by definition contain either an RNA or DNA genome surrounded by a protective, virus-coded protein coat. Viruses may be viewed as mobile genetic elements, most probably of cellular origin and characterized by a long co-evolution of virus and host. Viruses are inert outside the host cell. For propagation viruses depend on specialized host cells supplying the complex metabolic and biosynthetic machinery of eukaryotic or prokaryotic cells.

A complete virus particle is called a virion. The main function of the virion is to deliver its DNA or RNA genome into the host cell so that the genome can be expressed (transcribed and translated) by the host cell. The simplest virions consist of two basic components: nucleic acid (single- or double-stranded RNA or DNA) and a protein coat, the capsid, which functions as a shell to protect the viral genome from nucleases and which during infection attaches the virion to specific receptors exposed on the prospective host cell. Capsid proteins are coded for by the virus genome. Because of the limited size, the virus genome codes for only a few structural proteins (besides non-structural regulatory proteins involved in virus replication). Capsids are formed as single or double protein shells and consist of only one or a few structural protein species. Therefore, multiple protein copies must self-assemble to form the continuous three-dimensional capsid structure. Self-assembly of virus capsids follows two basic patterns: helical symmetry, in which the protein subunits and the nucleic acid are arranged in a helix, and icosahedral symmetry, in which the protein subunits assemble into a symmetric shell that covers the nucleic acid-containing core. The nucleic acid-associated protein, called nucleoprotein, together with the genome, forms the nucleocapsid. In enveloped viruses, the nucleocapsid is surrounded by a lipid bilayer derived from the modified host cell membrane and studded with an outer layer of virus envelope glycoproteins.

Viruses are grouped at different hierarchical levels of order, family, subfamily, genus and species. More than 30,000 different virus isolates are known and grouped in more than 3,600 species, in 164 genera, and 71 families. Viral morphology provides the basis for grouping viruses into families. A virus family may consist of members that replicate only in vertebrates, only in invertebrates, only in plants, or only in bacteria. Certain families contain viruses that replicate in more than one of these hosts.

Besides physical properties, several factors pertaining to the mode of replication play a role in classification: the configuration of the nucleic acid (i.e., single stranded or double stranded, linear or circular), whether the genome consists of one molecule of nucleic acid or is segmented, and whether the strand of ss RNA is sense or antisense. Also considered in classification is the site of viral capsid assembly and, in enveloped viruses, the site of nucleocapsid envelopment.

The use of Latinized names ending in -viridae for virus families and ending in -virus for viral genera has gained wide acceptance and is the method used herein. The names of subfamilies as used herein end in -virinae. Vernacular names are used herein to describe the viruses within a genus.

In the context of the instant disclosure, viruses suitable for use herein (e.g., to supply the surface glycoproteins, and/or to provide the virion or VLP framework) are selected from the following families: phycodnaviridae, flaviviridae, amnoonviridae, Orthomyxoviridae, asfarviridae, bacilladnaviridae, turriviridae, hepadnaviridae, arenaviridae, cruliviridae, discoviridae, fimoviridae, hantaviridae, leishbuviridae, mypoviridae, nairoviridae, peribunyaviridae, phasmaviridae, phenuiviridae, tospoviridae, tulasviridae, wupedeviridae, posviridae, circoviridae, vilyaviridae, ceraviridae, intestiviridae, steigviridae, suoliviridae, smacoviridae, mitoviridae, amalgaviridae, curvulaviridae, fusariviridae, hypoviridae, partitiviridae, picobirnaviridae, beminiviridae, genomoviridae, chrysoviridae, megabirnaviridae, quadriviridae, totiviridae, yueviridae, matshushitaviridae, simuloviridae, sphaerolipoviridae, pleolipoviridae, alphatetraviridae, benyviridae, hepeviriridae, matonaviridae, alloherpesviridae, Herpesviridae, malacoherpesviridae, mimiviridae, aliusviridae, chuviridae, crepuscuviridae, myriavitidae, natareviridae, tectiviridae, graaviviridae, haloferuviridae, pyrstoviridae, shortaselviridae, baculoviridae, hytrosaviridae, nuciviridae, lipothrixviridae, lipothrixviridiae, rudiviridae, ungulaviridae, bromoviridae, closteroviridae, endornaviridae, kitaviridae, mayoviridae, togaviridae, virgaviridae, anaeodiviridae, leisingerviridae, cystoviridae, artoviridae, bornaviridae, filoviridae, lispiviridae, mymonaviridae, nyamiviridae, paramyxoviridae, pneumoviridae, rhabdoviridae, sunviridae, xinmoviridae, metaxyvifidae, nanoviridae, qinviridae, abyssoviridae, arteriviridae, cremegaviridae, gresnaviridae, olifoviridae, coronaviridae, medioniviridae, mesoniviridae, mononiviridae, nanghoshaviridae, nanhypoviridae, euroniviridae, roniviridae, tobaniviridae, nodaviridae, sinhaliviridae, atkinsviridae, duinviridae, fiersviridae, solspiviridae, belpaoviridae, caulimoviridae, metaviridae, pseudoviridae, reroviridae, adintoviridae, botourmiaviridae, potyviridae, microviridae, dicistroviridae, iflaviridae, marnaviridae, picornaviridae, polycipiviridae, secoviridae, ascoviridae, iridoviridae, marseilleviridae, bidnaviridae, lavidaviridae, tristromaviridae, redondoviridae, sedoreoviridae, spinareoviridae, naryaviridae, nenyaviridae, adenoviridae, polyomaviridae, aspiviridae, alvernaviridae, barnaviridae, solemoviridae, astroviridae, druskaviridae, hafunaviridae, halomagnusviridae, soleiviridae, blumeviridae, steitzviridae, carmotetraviridae, tombusviridae, inoviridae, paulinoviridae, plectroviridae, alphaflexiviridae, betaflexiviridae, deltaflexiviridae, gammaflexiviridae, tymoviridae, corticoviridae, narnaviridae, yadokariviridae, papillomaviridae, ackermannviridae, aggregaviridae, alphasatellitidae, ampullaviridae, anelloviridae, assiduviridae, autographiviridae, autolykivifidae, avsunviroidae, bicaudaviridae, birnaviridae, casjensviridae, chaseviridae, clavaviridae, demerecviridae, drexlerviridae, duneviridae, finnlakeviridae, forsetiviridae, globuloviridae, fuselloviridae, guelinviridae, guttaviridae, hadakaviridae, halspiviridae, helgolandviridae, herelleviridae, kolmioviridae, kyanoviridae, madisaviridae, mesyanzhinovviridae, molycolviridae, naomviridae, minaviridae, orlajensenviridae, ovaliviridae, pachyviridae, peduoviridae, permutotetraviridae, pervagoviridae, plasmaviridae, polydnaviriformidae, polymycoviridae, portogloboviridae, pospiviroidae, rountreeviridae, salasmaviridae, saparoviridae, sarthroviridae, schitoviridae, spiraviridae, straboviridae, suolaviridae, thaspiviridae, tolecusatellitidae, vertoviridae, vilmaviridae, winoviridae, yaraviridae, zierdtviridae, and zobellviridae.

In some embodiments of the disclosure, viruses are selected from the following families: parvoviridae, papovaviridae, adenoviridae, herpesviridae, poxviridae, hepadnaviridae, picornaviridae, caliciviridae, astroviridae, togaviridae, flaviviridae, reoviridae, orthomyxoviridae, paramyxoviridae, rhabdoviridae, bunyaviridae, coronaviridae, arenaviridae, retroviridae, and filoviridae.

In some embodiments of the disclosure, viruses are selected from the following genera: filoviruses, matonaviruses, coronaviruses, arenaviruses, orthomyxoviruses, retroviruses, flaviviruses, togaviruses, alphaviruses, and paramyxoviruses.

In some embodiments of the disclosure, the viruses are selected from Nipah virus (NiV), Hendra virus (HeV), Ebola virus (EBOV), Rift valley fever virus (RVFV), peste des petits ruminants virus (PPRV), sheeppox virus (SPPV) or goat pox virus (GTPV), influenzas, and beta-coronaviruses.

Paramyxoviruses such as HeV and NiV possess two major membrane-anchored glycoproteins in the envelope of the viral particle. One glycoprotein is required for virion attachment to receptors on host cells and is designated as either hemagglutinin-neuraminidase protein (HN) or hemagglutinin protein (H), and the other is glycoprotein (G), which has neither hemagglutination nor neuraminidase activities. The attachment glycoproteins are type II membrane proteins, where the molecule's amino (N) terminus is oriented toward the cytoplasm and the protein's carboxy (C) terminus is extracellular. The other major glycoprotein is the fusion (F) glycoprotein, which is a trimeric class I fusogenic envelope glycoprotein containing two heptad repeat (HR) regions and a hydrophobic fusion peptide. HeV and NiV infect cells though a pH-independent membrane fusion process into receptive host cells through the concerted action of their attachment G glycoprotein and F glycoprotein following receptor binding. The primary function of the HeV and NiV attachment G glycoprotein is to engage appropriate receptors on the surfaces of host cells, which for the majority of well-characterized paramyxoviruses are sialic acid moieties. The HeV and NiV G glycoproteins utilize the host cell protein receptors ephrin B2 and/or ephrin B3 and antibodies have been developed which block viral attachment by the G glycoprotein (WO2006137931, Bishop (2008) J. Virol. 82: 11398-11409).

The filoviruses (e.g. Ebola Zaire 1976) cause acute hemorrhagic fever characterized by high mortality. Humans can contract filoviruses by infection in endemic regions, by contact with imported primates, and by performing scientific research with the virus. However, there currently are no available vaccines or effective therapeutic treatments for filovirus infection. The virions of filoviruses contain seven proteins: a membrane-anchored glycoprotein (GP), a nucleoprotein (NP), an RNA-dependent RNA polymerase (L), and four virion structural proteins (VP24, VP30, VP35, and VP40).

Rift Valley fever virus (RVFV) causes sporadic but devastating outbreaks of severe human disease and widespread morbidity and mortality in livestock. RVFV is a mosquito-borne virus of the family Bunyaviridae and genus Phlebovirus, and the timing of outbreaks is often closely associated with emergence of floodwater Aedes species mosquitoes following periods of extensive heavy rainfall. Although so far confined to Africa and the Arabian Peninsula, RVFV has the potential to spread to other parts of the world, given the presence and changing distribution of competent vectors throughout Europe and the Americas. Livestock (e.g., sheep, cattle and goats) are particularly susceptible to RVFV disease; outbreaks are characterized by widespread abortion storms and neonatal mortality approaching 100%. Swanepoel and Coetzer, Rift Valley fever, p. 688-717. In J. A. W. Coetzer, G. R. Thomson, and R. C. Tustin (ed.), Infectious Diseases of Livestock with Special Reference to South Africa. Oxford University Press, Cape Town, 1994. Infection in adult animals is associated with lower mortality, but the loss of a large proportion of young animals has serious economic impact. Humans usually become infected after handling aborted materials or other infected animal tissues, or through the bite of an infected mosquito.

RVFV has a tripartite negative-sense single-stranded RNA genome composed of three genome segments, S, M and L. The large (L) segment encodes the viral polymerase. The medium (M) segment encodes the structural glycoproteins, Gn and Gc, as well as non-structural proteins, including a 78 kD protein and NSm, a virulence factor suggested to function by inhibiting apoptosis. Won et al., J. Virol. 81:13335-13345, 2007. The ambisense small (S) segment encodes, in the viral sense, the nucleoprotein (NP) that is required for RNA synthesis, and the non-structural NSs protein in the opposite orientation. NSs is the major RVFV virulence factor and functions to inhibit the host immune response (Bouloy et al., J. Virol. 75:1371-1377, 2001) by generalized downregulation of host transcription (Billecocq et al., J. Virol. 78:9798-9806, 2004; Le May et al., Cell 116:541-550, 2004), post-transcriptional degradation of protein kinase R (PKR) (Habjan et al., J. Virol. 83:4365-4375, 2009; Ikegami et al., Ann. N. Y. Acad. Sci. 1171 Suppl 1:E75-85, 2009), and repression of the interferon-0 (IFN-0) promoter (Le May et al., PLoS Pathogens 4:e13, 2008). RVF virus structural proteins include nucleoprotein (NP or N, used interchangeably), two glycoproteins (Gn and Gc) and the viral RNA-dependent RNA polymerase (L protein).

Peste des petits ruminants virus (PPRV) causes a highly infectious disease of small ruminants that is endemic across Africa, the Middle East and large regions of Asia. PPRV is considered to be a major obstacle to the development of sustainable agriculture across the developing world. The disease is emerging in new regions of the world and is causing significant economic losses. PPRV belongs to the genus Morbillivirus of the family Paramyxoviridae, alongside other important viral pathogens, e.g., Rinderpest virus, Measles virus, Canine distemper virus, Phocine distemper virus and the morbilliviruses of marine mammals, the cetacean morbilliviruses. The PPRV viral envelope is derived from infected cell membrane, and is studded with glycoprotein peplomers consisting of the viral fusion (F) and haemagglutinin (H) glycoproteins. The PPRV genome consists of a non-segmented, single-stranded, negative-sense RNA molecule encapsidated by nucleoprotein (N) forming a helical nucleocapsid, in combination with the RNA-dependent RNA polymerase (L; large polymerase) and the co-factor phosphoprotein (P; polymerase complex) to form the ribonucleoprotein (RNP) complex. Ribonucleoproteins are located within the virus envelope and appear as helices with a herringbone appearance. The matrix protein (M) located on the inner surface of the envelope bridges the ribonucleoprotein and cytoplasmic tails of the membrane glycoproteins.

Sheeppox virus (SPPV) and goatpox virus (GTPV), members of the Capripoxvirus genus of the Poxviridae, are etiologic agents of important diseases of sheep and goats in northern and central Africa, southwest and central Asia, and the Indian subcontinent. SPPV and GTPV are endemic throughout southwest and central Asia, the Indian subcontinent, and northern and central Africa.

Influenza viruses are one of the most ubiquitous viruses present in the world, affecting both humans and livestock. Influenza results in an economic burden, morbidity and even mortality, all of which are significant. Influenza viruses cause epidemics almost every winter, with infection rates for type A or B virus as high as 40% over a six-week period. Influenza infection results in various disease states, from a sub-clinical infection through mild upper respiratory infection to a severe viral pneumonia. Typical influenza epidemics cause increases in incidence of pneumonia and lower respiratory disease as witnessed by increased rates of hospitalization or mortality. The severity of the disease is primarily determined by the age of the host, his immune status and the site of infection.

The influenza virus is an RNA enveloped virus with a particle size of about 125 nm in diameter. It consists basically of an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material. Influenza virus comprises two surface antigens, glycoproteins neuraminidase (NA) and haemagglutinin (HA), which appear as spikes, 10 to 12 nm long, at the surface of the particles. It is these surface proteins, particularly the haemagglutinin that determine the antigenic specificity of the influenza subtypes. Virus strains are classified according to host species of origin, geographic site and year of isolation, serial number, and, for influenza A, by serological properties of subtypes of HA and NA. 16 HA subtypes (H1-H16) and nine NA subtypes (N1-N9) have been identified for influenza A viruses. Webster R G et al. Evolution and ecology of influenza A viruses. Microbiol. Rev. 1992; 56:152-179; Fouchier R A et al. Characterization of a Novel Influenza A Virus Hemagglutinin Subtype (H16) Obtained from Black-Headed Gulls. J. Virol. 2005; 79:2814-2822. Viruses of all HA and NA subtypes have been recovered from aquatic birds, but only three HA subtypes (H1, H2, and H3) and two NA subtypes (N1 and N2) have established stable lineages in the human population since 1918. Only one subtype of HA and one of NA are recognised for influenza B viruses.

Several coronaviruses have led to serious diseases in humans. These include SARS-CoV-1), Middle east respiratory syndrome-CoV (MERS-CoV) and most recently SARS-CoV-2 that has led to a global pandemic. Coronavirus particles are composed of 4 structural proteins including spike (S) protein, envelope (E) protein, membrane (M) protein, and nucleoprotein (N). Coronaviruses (CoVs) are enveloped positive-sense RNA viruses. Enveloped CoVs entering host cells and initiating infection is achieved through the fusion of viral and cellular membranes. Membrane fusion is mediated by the large type I transmembrane S glycoprotein on the viral envelope and the cognate receptor on the surface of host cells.

Many viruses have variants or strains. As used herein, a variant is a viral genome that may contain one or more mutations from the original virus. A strain is a genetic variant that has distinct physical properties different from the reference or wild type virus. A number of RVF virus strains have been identified.

Influenza A viruses evolve and undergo antigenic variability continuously. Wiley D, Skehel J. The structure and the function of the hemagglutinin membrane glycoprotein of influenza virus. Ann. Rev. Biochem. 1987; 56:365-394. A lack of effective proofreading by the viral RNA polymerase leads to a high rate of transcription errors that can result in amino-acid substitutions in surface glycoproteins, termed “antigenic drift.” The segmented viral genome allows for a second type of antigenic variation. If two influenza viruses simultaneously infect a host cell, genetic reassortment, called “antigenic shift” may generate a novel virus with new surface or internal proteins. These antigenic changes, both ‘drifts’ and ‘shifts’ are unpredictable and may have a dramatic impact from an immunological point of view as they eventually lead to the emergence of new influenza strains and that enable the virus to escape the immune system causing the well-known, almost annual, epidemics. Both of these genetic modifications have caused new viral variants responsible for pandemic in humans.

A viral protein is both a component and a product of a virus. Viral proteins are grouped according to their functions, and groups of viral proteins include structural proteins, nonstructural proteins, regulatory proteins, and accessory proteins. A “structural” protein is a protein found in the virus particle, whereas a “non-structural” protein is only expressed in a virus-infected cell.

The genetic material of a virus is stored within a viral protein structure called the capsid. The capsid is a “shield” that protects the viral nucleic acids from getting degraded by host enzymes or other types of pesticides or pestilences. The capsid of some viruses is enclosed in a membrane called the viral envelope. In most cases, the viral envelope is obtained by the capsid from the host cell's plasma membrane when a virus leaves its host cell through a process called budding. The viral envelope is made up of a lipid bilayer embedded with viral proteins, including viral glycoproteins. These viral glycoproteins bind to specific receptors and coreceptors on the membrane of host cells, and they allow viruses to attach onto their target host cells. Viral glycoproteins play a critical role in virus-to-cell fusion. Virus-to-cell fusion is initiated when viral glycoproteins bind to cellular receptors.

Viral proteins which mediate the attachment and entry of the virus into the susceptible host cell are described herein as receptor binding glycoproteins. Examples of such proteins include the G protein of NiV. Additional examples include the envelope glycoprotein of West Nile Virus.

Membrane fusion is critical for viral entry. Viral fusion proteins cause fusion of biological membranes. Enveloped viruses readily overcome the thermodynamic barrier of merging two plasma membranes by storing kinetic energy in fusion (F) proteins. F proteins can be independently expressed on host cell surfaces which can either (1) drive the infected cell to fuse with neighboring cells, forming a syncytium, or (2) be incorporated into a budding virion from the infected cell which leads to the full emancipation of plasma membrane from the host cell. Some F components solely drive fusion while a subset of F proteins can interact with host factors.

### Multivalent Viral Particles

In one aspect, the disclosure is directed to a composition comprising a population of multivalent viral particles. Such a composition is a grouping, or population, of virus derived structures (e.g., replication incompetent virions or VLPs) which are capable of mimicking the form and size of a virus particle but lack the necessary genetic material and are therefore unable to infect a subject.

As used herein, “a population” of viral particles is an assembly or preparation of viral particles.

In some embodiments, the population of particles can be heterogenous or homogenous. As used herein a “heterogenous population” is a population of particles displaying different surface glycoproteins (e.g., with some particles displaying surface glycoproteins from two viruses, and other particles displaying surface glycoproteins from three or more viruses), while a “homogenous population” is a population of particles displaying the same surface glycoproteins. The glycoprotein composition is used herein to describe the types of glycoproteins on the surface of the particles. The glycoprotein composition will not always be the exact same number of each type of glycoprotein. Variations can and do occur. However, in some embodiments, the population of particles will be comprised of at least 80% of particles having substantially the same surface glycoprotein composition.

In some embodiments, the population of particles is purified. The term “purified” does not require absolute purity; rather, it is intended as a relative term. In some embodiments, the population is purified and substantially homogeneous in their surface glycoproteins; e.g., the population is composed predominantly multivalent viral particles displaying surface glycoproteins from two viruses, or predominantly multivalent viral particles displaying surface glycoproteins from three viruses, or the like. By “predominantly” it is meant at least 75%, at least 80%, at least 85%, at least 90%, at least 95%, or greater of the particles in the population display the same types of surface glycoproteins.

The population of particles can be described as a number of particles in the population. In some embodiments, the number of particles in a population is more than 10,000 particles. In some embodiments, the number of particles in a population is more than 20,000 particles. In some embodiments, the number of particles in a population is more than 30,000 particles. In some embodiments, the number of particles in a population is more than 40,000 particles. In some embodiments, the number of particles in a population is more than 50,000 particles. In some embodiments, the number of particles in a population is more than 100,000 particles. In some embodiments, the number of particles in a population is more than 250,000 particles. In some embodiments, the number of particles in a population is more than 500,000 particles. In some embodiments, the number of particles in a population is more than 750,000 particles. In some embodiments, the number of particles in a population is more than 1,000,000 particles. In some embodiments, the number of particles in a population is more than 10,000,000 particles. In some embodiments, the number of particles in a population is more than 50,000,000 particles. In some embodiments, the number of particles in a population is more than 100,000,000 particles. In some embodiments, the number of particles in a population is more than 200,000,000 particles. In some embodiments, the number of particles in a population is more than 300,000,000 particles. In some embodiments, the number of particles in a population is more than 400,000,000 particles. In some embodiments, the number of particles in a population is more than 500,000,000 particles. In some embodiments, the number of particles in a population is more than 600,000,000 particles. In some embodiments, the number of particles in a population is more than 700,000,000 particles. In some embodiments, the number of particles in a population is more than 800,000,000 particles. In some embodiments, the number of particles in a population is more than 900,000,000 particles. In some embodiments, the number of particles in a population is more than 1,000,000,000 particles.

In some embodiments, the viral particles are replication incompetent virions. In some embodiments, the viral particles are VLPs. VLPs are virus-derived structures made up of one or more different molecules with the ability to self-assemble, mimicking the form and size of a virus particle but lacking the genetic material so they are not capable of infecting the host cell. Expression and self-assembly of the viral structural proteins can take place in various living or cell-free expression systems after which the viral structures can be assembled and reconstructed. VLPs are highly immunogenic and are able to elicit both the antibody- and cell-mediated immune responses by pathways different from those elicited by conventional inactivated viral vaccines. VLPs can be produced in a variety of systems, including mammals, plants, insects, and bacteria. VLPs are icosahedral or rod-shaped structures made by the self-assembly of viral structural proteins. Due to their size and shape, which resembles the actual size and shape of native viruses, these structures can efficiently elicit the immune responses and in VLPs lacking viral genomes there is no potential for replication within the target cells, which offers improved safety especially for immunocompromised or elderly vaccines. In general, the process of manufacturing a VLP-based vaccine consists of three stages. 1) a Production stage which includes cloning of the viral structural genes of interest and expression of viral proteins with self-assembling ability in a suitable expression platform (the HEK293T cell line, a mammalian expression system, is one non-limiting example). At the end of the production stage, the VLPs are collected in the form of particles that do not have infectious properties. 2) a Purification stage which briefly consist of downstream processing such as clarification, purification and polishing to obtain purified intact VLPs with no residual host debris. 3) a Formulation stage, in which adjuvant and additional ingredients are added to the vaccine formulation to finally achieve a safe, efficient, and effective product for vaccination.

A replicon is equivalent to a full-length virus from which all of the viral structural proteins have been deleted. A multiple cloning site can be inserted downstream of the 26S promoter into the site previously occupied by the structural protein genes. Virtually any heterologous gene may be inserted into this cloning site. The RNA that is transcribed from the replicon is capable of replicating and expressing viral proteins in a manner that is similar to that seen with the full-length infectious virus clone. However, in lieu of the viral structural proteins, the heterologous antigen is expressed from the 26S promoter in the replicon. Such a system does not yield any progeny virus particles because there are no viral structural proteins available to package the RNA into particles.

Particles which appear structurally identical to virus particles can be produced by supplying structural protein RNAs in trans for packaging of the replicon RNA. This is typically done with two defective helper RNAs which encode the structural proteins. One helper consists of a full-length infectious clone from which the nonstructural protein genes and the glycoprotein genes are deleted. This helper retains only the terminal nucleotide sequences, the promoter for subgenomic mRNA transcription and the sequences for the viral nucleocapsid protein. The second helper is identical to the first except that the nucleocapsid gene is deleted and only the glycoprotein genes are retained. The helper RNAs are transcribed in vitro and are co-transfected with replicon RNA. Because the replicon RNA retains the sequences for packaging by the nucleocapsid protein, and because the helpers lack these sequences, only the replicon RNA is packaged by the viral structural proteins. The packaged replicon particles are released from the host cell and can then be purified and inoculated into animals. The packaged particles will have a tropism similar to the parent virus. The packaged replicon particles will infect cells and initiate a single round of replication, resulting in the expression of only the virus nonstructural proteins and the product of the heterologous gene that was cloned in the place of the virus structural proteins. In the absence of RNA encoding the virus structural proteins, no progeny virus particles can be produced from the cells infected by packaged replicon particles.

Replication incompetent viruses are specifically defective for viral functions that are essential for viral genome replication and assembly of progeny virus particles. They are propagated in complementing cell lines that express the missing viral gene product(s), allowing viral replication. In contrast, in normal cells, one or more steps in viral replication are blocked, resulting in viral gene expression within the infected cell but no progeny virus production. There are several examples of replication incompetent virions that are commonly used in the art, including but not limited to, virus like particles (VLP) and pseudotyped viruses. Pseudotyping, in its original sense, means that one or more of the structural proteins of a virus particle are not encoded by the nucleic acid carried by the virus. Using this broad definition, pseudotyped viruses include any recombinant viral gene transduction system that is dependent for genome packaging upon helper proteins expressed from defective genomes in the viral producer cells or upon infection by a helper virus. The common current usage in the gene-transfer field dictates that a pseudotyped virus is one in which the outer shell (the envelope glycoproteins of an enveloped virus or the capsid proteins of a nonenveloped virus) originates from a virus that differs from the source of the genome and the genome replication apparatus. Pseudotyped viruses (PVs) are replication-defective viral particles formed with a structural and enzymatic core from one virus and the envelope glycoprotein of another. For example, constructing a chimeric glycoprotein where the cytoplasmic domain of the G protein of one virus is switched for that of another. Such PVs have little associated risk of infectivity. One popular PV is the vesicular stomatitis virus (VSV) that has been produced expressing chimeric envelope glycoproteins.

In accordance with this disclosure, the viral particles in the population each comprise (i) a matrix glycoprotein base, wherein the matrix glycoprotein is from a first virus species, and (ii) surface glycoproteins from at least two virus species.

Viral matrix proteins are structural proteins linking the viral envelope with the virus core. They play a crucial role in virus assembly and interact with the ribonucleoprotein complex as well as with the viral membrane. They are found in many enveloped viruses including paramyxoviruses, orthomyxoviruses, herpesviruses, retroviruses, filoviruses, among others. Viral matrix proteins, like many other viral proteins, can exert different functions during the course of the infection. For example, in rhabdoviruses, binding of M proteins to nucleocapsids is accountable for the formation of its “bullet” shaped virions. Another example is the M1 protein of the influenza virus. Since the matrix proteins link the viral envelope with the virus core, the matrix proteins are able to organize the viral particles. The viral matrix protein can create the spontaneous interaction between one or more viral structural capsid proteins to form the final particle structure. In other words, the viral matrix protein is responsible for spontaneously forming the particle when the surface glycoproteins are present.

In some embodiments, the matrix protein of the multivalent viral particles is selected from a virus in one of the following families: paramyxovirus, filovirus, flavivirus, orthomyxovirus, arenavirus, retrovirus, coronavirus, rhabdoviruses, togavirus, and alphavirus. In some embodiments, the matrix protein is from a virus in the paramyxovirus family. In some embodiments, the matrix protein is a Nipah virus matrix protein (GenBank accession number NP_112025, see Table 1). In some embodiments, the matrix protein is a Hendra virus matrix protein (GenBank accession number NP_047110, see Table 1).

In some embodiments, the viral surface glycoproteins which make up the outer portion of the particle, or that resemble the viral envelope, are selected from GP proteins, spike proteins, amino-terminus glycoproteins (Gn), carboxyl-terminus glycoprotein (Gc), neuraminidase (NA), hemagglutinin (H), and/or P32 protein. In some embodiments, the viral surface glycoproteins include spike proteins of the coronaviruses. In some embodiments, the viral surface glycoproteins include SARS-CoV-1 spike protein, SARS-CoV-2 spike protein, and MERS-CoV spike protein. In some embodiments, the viral surface glycoproteins include neuraminidase from an influenza virus. In some embodiments, the viral surface glycoproteins include RVFV GnGc, PPRV F and H, and GTPV P32. In some embodiments, the viral surface glycoproteins include NiV-F, NiV-G, HeV-F, HeV-G, and EBOV-GP. See Table 1 for GenBank accession numbers.

TABLE 1

| Name of Protein | GenBank accession number |
|---|---|
| NiV F | NP_112026 |
| NiV G | NP_112027 |
| NiV M | NP_112025 |
| HeV F | NP_047111 |
| HeV G | NP_047112 |
| HeV M | NP_047110 |
| EBOV GP | NP_066246 |
| SARS-CoV1 Spike | P59594 |
| SARS-CoV2 Spike | BCN86353 |
| MERS CoV Spike | AHX71946 |
| RVFV Gn/Gc | QUE40317 |
| PPRV F | AAL78974 |
| PPRV H | AGG09146 |
| GTPV P32 | QXV24334 |
| influenza neuraminidase | P03468 |

Some embodiments of the disclosure are directed to a population of particles comprising surface glycoproteins of at least 2 species of viruses, wherein one of the 2 virus species supplies the matrix glycoprotein. In some embodiments, the population of particles comprising surface glycoproteins of at least 2 species of viruses, include a virus species different from the virus species supplying the matrix glycoprotein. In some embodiments, the population of particles comprising surface glycoproteins of at least 2 species of viruses, include virus species from two virus families. In some embodiments, the population of particles is a population of virus like particles comprising surface glycoproteins of at least 2 species of viruses. In some embodiments, the population of particles is a population of pseudovirus particles comprising surface glycoproteins of at least 2 species of viruses. In some embodiments, the population of particles is a population of replication-incompetent virions comprising surface glycoproteins of at least 2 species of viruses. In some embodiments, the virion is a pseudotyped vesicular stomatitis virus (VSV). In some embodiments, the pseudotyped VSV is a recombinant VSV wherein the native envelope G protein is removed. In some embodiments, the pseudotyped VSV is a recombinant VSV wherein the native G protein is replaced by a reporter protein.

In some embodiments of the disclosure, the population of particles comprises surface glycoproteins of at least 2 species of viruses, wherein the first virus species is selected from Nipah (NiV), Hendra (HeV), Ebola (EBOV), Rift valley fever (RVFV), peste des petits ruminants (PPRV), goat pox (GTPV), influenzas, and beta-coronaviruses. In some embodiments, the beta-coronaviruses are SARS-CoV-1, SARS-CoV-2, MERS-CoV, and their variants.

Some embodiments of the disclosure are directed to a population of particles comprising surface glycoproteins of at least 2 species of viruses. In some embodiments, the population of particles comprising surface glycoproteins of at least 2 species of viruses include a virus species different from the first virus species. In some embodiments, the population of particles is a population of virus like particles comprising surface glycoproteins of at least 2 species of viruses. In some embodiments, the population of particles is a population of pseudovirus particles comprising surface glycoproteins of at least 2 species of viruses. In some embodiments, the population of particles is a population of replication-incompetent virions comprising surface glycoproteins of at least 2 species of viruses. In some embodiments, the virion is a pseudotyped vesicular stomatitis virus (VSV). In some embodiments, the pseudotyped VSV is a recombinant VSV wherein the native envelope G protein is removed. In some embodiments, the pseudotyped VSV is a recombinant VSV wherein the native G protein is replaced by a reporter protein.

In some embodiments of the disclosure, the population of particles comprises surface glycoproteins of at least 2 species of viruses, wherein the first virus species is selected from Nipah (NiV), Hendra (HeV), Ebola (EBOV), Rift valley fever (RVFV), peste des petits ruminants (PPRV), goat pox (GTPV), influenzas, and beta-coronaviruses. In some embodiments, the beta-coronaviruses are SARS-CoV-1, SARS-CoV-2, MERS-CoV, and their variants. In some embodiments, the population of particles comprises surface glycoproteins of at least 2 species of viruses, wherein the surface glycoproteins are selected from NiV-F, NiV-G, HeV-F, HeV-G, and EBOV-GP. In some embodiments, the population of particles comprises surface glycoproteins of at least 2 species of viruses, wherein the surface glycoproteins are selected from SARS-CoV-1 spike protein, SARS-CoV-2 spike protein, and MERS-CoV spike protein. In some embodiments, the population of particles comprises surface glycoproteins of at least 2 species of viruses, wherein the surface glycoproteins are selected from RVFV GnGc, PPRV F and H, and GTPV P32. In some embodiments, the population of particles comprises surface glycoproteins of at least 2 species of viruses, wherein the surface glycoproteins are selected from SARS-CoV-2 spike protein and influenza neuraminidase (NA).

Some embodiments of the disclosure are directed to a population of particles comprising surface glycoproteins of at least 3 species of viruses. Some embodiments of the disclosure are directed to a population of particles comprising surface glycoproteins from more than 3 species of viruses.

In some embodiments, the corresponding surface glycoproteins are selected from NiV-F glycoprotein, NiV-G glycoprotein, HeV-F glycoprotein, HeV-G glycoprotein, EBOV-GP glycoprotein, SARS-CoV-1 spike protein, SARS-CoV-2 spike protein, MERS-CoV spike protein, RVFV GnGc glycoproteins, PPRV F and H glycoproteins, GTPV P32 glycoproteins, and/or influenza neuraminidase (NA) glycoprotein. In some embodiments, the corresponding surface glycoproteins are selected from NiV-F, NiV-G, HeV-F, HeV-G, and EBOV-GP. In some embodiments, the corresponding surface glycoproteins are selected from SARS-CoV-1 spike protein, SARS-CoV-2 spike protein, and MERS-CoV spike protein. In some embodiments, the corresponding surface glycoproteins are selected from RVFV GnGc, PPRV F and H, and GTPV P32. In some embodiments, the corresponding surface glycoproteins are selected from SARS-CoV-2 spike protein and influenza neuraminidase (NA).

In some embodiments of the disclosure, the particles comprise reporter proteins. As used herein, a “reporter protein” is an easily detected protein that does not present normally in the particle. Reporter proteins can be used to track location and to assure that the experimental design is working properly. Examples of reporter proteins are green fluorescent protein, biotin, luciferase, chloramphenyl acetyltransferase, β-galactosidase, red fluorescent protein, flag tag, maltose binding protein, glutathione s-transferase, V5 tag, myc, HA, and His, and AU1. Some of the reporter proteins can be visually seen when expressed by cells using fluorescent filter microscopy. Other reporter proteins can be detected through immunoblotting. In some embodiments, the protein constructs had the following DNA tags: NiV M-flag, NiV F-flag, NiV G-myc, HeV F-AU1, HeV G-HA and EBOV GP-V5.

Lyophilized formulations are one of the methods for preparation of an immunogenic composition. Lyophilized preparations of immunogenic compositions of the current disclosure typically contain purified virus glycoproteins and at least carbohydrate. In some embodiments, the carbohydrate is sugar polyol. In some embodiments, the at least one carbohydrate is sucrose or trehalose. In some embodiments, the at least one sugar polyol is sorbitol. In some embodiments, the carbohydrate is in combination with a glass forming sugar. In some embodiments, the glass forming sugar is a disaccharide or an oligosaccharide. The disaccharide can be selected from the following list but is not limited to sucrose, trehalose, maltose, mannose, lactose, raffinose, isomaltose, stachyose, etc. Any one of ordinary skill in the art will devise further embodiments based on the disclosures above.

The lyophilized formulations can be re-suspended in water for injection or an aqueous buffer that is pharmaceutically acceptable for administration. e.g. as an injectable liquid to a subject. The lyophilized formulation can also be used as an inhalable powder which will be suitable for inducing mucosal immunity. Additionally, the lyophilized formulation of can comprise an adjuvant that confers mucosal immunity preferably from a list of those adjuvants tested and known in the art.

In some embodiments of the disclosure, the carbohydrate is at a concentration of 1%, 2%, 3%, 4%, 5%, 6%, 7%, 8%, 9%, or 10% wt. In some embodiments of the disclosure, the carbohydrate is at a concentration of 0.5%. In some embodiments of the disclosure, the carbohydrate is at a concentration of 1%. In some embodiments of the disclosure, the carbohydrate is at a concentration of 1.5%. In some embodiments of the disclosure, the carbohydrate is at a concentration of 2%. In some embodiments of the disclosure, the carbohydrate is at a concentration of 2.5%. In some embodiments of the disclosure, the carbohydrate is at a concentration of 3%. In some embodiments of the disclosure, the carbohydrate is at a concentration of 3.5%. In some embodiments of the disclosure, the carbohydrate is at a concentration of 4%. In some embodiments of the disclosure, the carbohydrate is at a concentration of 4.5%. In some embodiments of the disclosure, the carbohydrate is at a concentration of 5% wt. In some embodiments of the disclosure, the carbohydrate is at a concentration of 5.5%. In some embodiments of the disclosure, the carbohydrate is at a concentration of 6%. In some embodiments of the disclosure, the carbohydrate is at a concentration of 6.5%. In some embodiments of the disclosure, the carbohydrate is at a concentration of 7%. In some embodiments of the disclosure, the carbohydrate is at a concentration of 7.5%. In some embodiments of the disclosure, the carbohydrate is at a concentration of 8%. In some embodiments of the disclosure, the carbohydrate is at a concentration of 8.5%. In some embodiments of the disclosure, the carbohydrate is at a concentration of 9%. In some embodiments of the disclosure, the carbohydrate is at a concentration of 9.5%. In some embodiments of the disclosure, the carbohydrate is at a concentration of 10% wt.

“Immunologically protective amount” or “immunologically effective amount” or “effective amount to produce an immune response” of an antigen is an amount effective to induce an immunogenic response in the recipient. The immunogenic response may be sufficient for diagnostic purposes or other testing, or may be adequate to prevent signs or symptoms of disease, including adverse health effects or complications thereof, caused by infection with a disease agent. Either humoral immunity or cell-mediated immunity or both may be induced. The immunogenic response of an animal to an immunogenic composition may be evaluated, e.g., indirectly through measurement of antibody titers, lymphocyte proliferation assays, or directly through monitoring signs and symptoms after challenge with wild type strain, whereas the protective immunity conferred by a vaccine can be evaluated by measuring, e.g., reduction in clinical signs such as mortality, morbidity, temperature number, overall physical condition, and overall health and performance of the subject. The immune response may comprise, without limitation, induction of cellular and/or humoral immunity. “Immunogenic” means evoking an immune or antigenic response. Thus, an “immunogenic composition” would be any composition that induces an immune response.

“Therapeutically effective amount” refers to an amount of an antigen or vaccine that would induce an immune response in a subject receiving the antigen or vaccine which is adequate to prevent or reduce signs or symptoms of disease, including adverse health effects or complications thereof, caused by infection with a pathogen, such as a virus or a bacterium. Humoral immunity or cell-mediated immunity or both humoral and cell-mediated immunity may be induced. The immunogenic response of a subject to a vaccine may be evaluated, e.g., indirectly through measurement of antibody titers, lymphocyte proliferation assays, or directly through monitoring signs and symptoms after challenge with wild type strain. The protective immunity conferred by a vaccine can be evaluated by measuring, e.g., reduction in clinical signs such as mortality, morbidity, temperature number, overall physical condition, and overall health and performance of the subject. The amount of a vaccine that is therapeutically effective may vary depending on the particular adjuvant used, the particular antigen used, or the condition of the subject, and can be determined by one skilled in the art.

In some embodiments of the disclosure, the immunogenic response is induced to specific antigens. In some embodiments of the disclosure, the specific antigens include NiV-F glycoprotein, NiV-G glycoprotein, HeV-F glycoprotein, HeV-G glycoprotein, EBOV-GP glycoprotein, SARS-CoV-1 spike protein, SARS-CoV-2 spike protein, MERS-CoV spike protein, RVFV GnGc glycoproteins, PPRV F and H glycoproteins, GTPV P32 glycoproteins, and/or influenza neuraminidase (NA) glycoprotein. In some embodiments, the immunogenic response is induced by any combination of NiV-F glycoprotein, NiV-G glycoprotein, HeV-F glycoprotein, HeV-G glycoprotein, EBOV-GP glycoprotein, SARS-CoV-1 spike protein, SARS-CoV-2 spike protein, MERS-CoV spike protein, RVFV GnGc glycoproteins, PPRV F and H glycoproteins, GTPV P32 glycoproteins, and/or influenza neuraminidase (NA) glycoprotein. In some embodiments of the disclosure, the immunogenic response is induced to NiV-F, NiV-G, HeV-F, HeV-G, and EBOV-GP glycoproteins. In some embodiments of the disclosure, the immunogenic response is induced to SARS-CoV-1 spike protein, SARS-CoV-2 spike protein, and/or MERS-CoV spike protein. In some embodiment of the disclosure, immunogenic response is induced to the RVFV GnGc, PPRV F and H, and GTPV P32 glycoproteins. In yet other embodiments of the disclosure, the immunogenic response is induced to SARS-CoV-2 spike protein and/or influenza neuraminidase (NA) glycoprotein.

The disclosed embodiments may be administered to a subject in need thereof. In some embodiments, the subject is a mammal. In some embodiments, the subject is a human. In some embodiments, the subject is a rodent. In some embodiments, the subject is a livestock animal. Livestock animals include all animals that are bred, reared, and kept for use and/or profit. Examples of livestock include, but are not limited to cattle, sheep, pigs, goats, horses, donkeys, mules; buffalo, oxen, llamas, camels, alpaca, and yak. In some embodiments, the subject is fowl, including but not limited to waterfowl, such as ducks, swans, and geese; game fowl, such as turkey or pheasant; or domesticated barnyard birds like chickens.

The target human population to vaccinate is the entire population, e.g. healthy young adults (e.g. aged 18-60), elderly (typically aged above 60) or infants/children. The target population may in particular be immuno-compromised. Immuno-compromised humans generally are less well able to respond to an antigen, in particular to an influenza antigen, in comparison to healthy adults.

In some embodiments of the disclosure, antibodies are generated against specific antigens. In some embodiments, the antibodies produced are generated against NiV-F glycoprotein, NiV-G glycoprotein, HeV-F glycoprotein, HeV-G glycoprotein, EBOV-GP glycoprotein, SARS-CoV-1 spike protein, SARS-CoV-2 spike protein, MERS-CoV spike protein, RVFV GnGc glycoproteins, PPRV F and H glycoproteins, GTPV P32 glycoproteins, and/or influenza neuraminidase (NA) glycoprotein. In some embodiments, the antibodies produced are generated against any combination of NiV-F glycoprotein, NiV-G glycoprotein, HeV-F glycoprotein, HeV-G glycoprotein, EBOV-GP glycoprotein, SARS-CoV-1 spike protein, SARS-CoV-2 spike protein, MERS-CoV spike protein, RVFV GnGc glycoproteins, PPRV F and H glycoproteins, GTPV P32 glycoproteins, and/or influenza neuraminidase (NA) glycoprotein. In some embodiments, the antibodies produced are generated against NiV-F, NiV-G, HeV-F, HeV-G, and EBOV-GP glycoproteins. In some embodiments, the antibodies produced are generated against SARS-CoV-1 spike protein, SARS-CoV-2 spike protein, and/or MERS-CoV spike protein. In some embodiments, the antibodies produced are generated against RVFV GnGc, PPRV F and H, and GTPV P32 glycoproteins. In some embodiments, the antibodies produced are generated against SARS-CoV-2 spike protein and influenza neuraminidase (NA).

In some embodiments of the disclosure, antibodies are generated against a virus species which is not the same as the first virus species, nor any virus species that supply the glycoproteins. In other words, some embodiments of the disclosure generate antibodies that neutralize a virus with moderate sequence similarities to the virus species that supply the surface glycoproteins. For example, cross-neutralization of a related henipavirus, Cedar virus (CedV) having moderate sequence similarities to NiV and HeV G and F is also neutralizable when only NiV and HeV G and F glycoproteins are presented. Therefore, the multivalent vaccination strategy is capable of yielding cross-protective antibodies against henipaviruses beyond NiV and HeV, justifying further use and optimization of this vaccination strategy towards the goal of broadly protective vaccines.

Another aspect of the disclosure is directed to an immunogenic composition comprising a population of particles as described herein and a pharmaceutically acceptable buffer. An embodiment of the current disclosure is the choice of pharmaceutically acceptable buffer throughout the bioprocess wherein the buffering agent is selected from a list consisting of any one or more of the following, but not limited to: phosphate buffer; citrate buffer; phosphate citrate buffer; borate buffer; tris(hydroxymethyl)aminomethane (Tris) containing buffer; succinate buffer; buffers containing glycine or histidine as one of the buffering agents. In some embodiments, phosphate buffer is used, wherein phosphate buffer is sodium phosphate buffer at concentration of 5 mM up to 200 mM of phosphate ions, preferably 10 mM to 100 mM phosphate buffer, most preferably 10 mM to 50 mM phosphate buffer of any pH above 6.50 to pH 9, preferably pH 6.8 to pH 7.8 is used for the upstream and downstream processes.

### Methods of Making Multivalent Immunogenic Composition

The generic manufacturing process for a particle-based vaccine generally consists of three main sections: (1) upstream processing (production), (2) downstream processing (purification), and (3) formulation. The first step in particle production is to clone the viral structural genes of interest, as identified herein. Next, viral structural proteins with self-assembling ability are expressed in prokaryotic (bacteria, yeast) or eukaryotic (baculovirus/insect cell, mammalian cell and plant) host cell expression systems. After harvesting and lysing the cells, a clarification step is performed to ensure removal of contaminating cell debris and aggregates. T Vicente, et al., Large-scale production and purification of VLP-based vaccines. J Invertebr Pathol. 2011; 107:S42-8; N Hillebrandt, et al., Integrated process for capture and purification of virus-like particles: enhancing process performance by cross-flow filtration. Front Bioeng Biotechnol. 2020; 8:489. In order to obtain intact and more purified particles, further purification steps such as ion-exchange chromatography and ultracentrifugation can be performed as needed. N Hillebrandt, et al., Front Bioeng Biotechnol. 2020; 8:489. A final purification step, called polishing, is used to remove the residual host cell proteins and nucleic acids. T Vicente, et al., J Invertebr Pathol. 2011; 107:S42-8; C Peixoto, et al., J Biotechnol. 2007; 127(3):452-61. In the last step of manufacturing process of VLPs vaccine development, sterile filtration and formulation is done to finally achieve a safe, efficient and effective product. T Vicente, et al., J Invertebr Pathol. 2011; 107:S42-8.

Downstream processing for particle purification is a crucial step to ensure suitable efficacy and safety for clinical use. In some embodiments disclosed herein, the particles are purified for use as candidate vaccine. In some particles produced using insect cell culture, the particles are released into the medium without the need for special measures. However, if the particle is not released effectively, cell lysis or an alternative extraction method may be required to disrupt the cells and release the particles into the extracellular medium. Purification is achieved by a combination of both physical and chemical methods. Physical methods include any of the following techniques but not limited to: ultracentrifugation, density gradient centrifugation, ultrafiltration, diafiltration and concentration using semi-permeable membranes with suitable molecular cut-off sizes. Purification through chemical means employs methods such as adsorption/desorption through chemical or physiochemical reactions such as ion exchange chromatography, affinity chromatography, hydrophobic interaction chromatography, gel filtration chromatography such as for example Captocore700™, hydroxyapatite matrix, salting with inorganic salts, one such example being ammonium sulphate.

In some embodiments, the particles are purified on Capto core 700 (GE Healthcare Life Sciences) column chromatography. The particle harvest before Capto core 700 column can be clarified using membrane filters with different pore sizes, preferably not less than 0.45 μM low protein binding membrane. In some embodiments, the particle harvest can be clarified with a dual membrane of two different pore sizes, for example 1.2 μM followed by 0.45 μM, or 0.8 μM followed by 0.45 μM. The clarified virus harvest is suitable for purification on Capto Core 700 column. The buffers used for purification on Capto core 700 is of optimal pH and ionic strength to maximize the binding of the impurities on the column and elute the virus in the flow through. The particle sample can be further concentrated by diafiltration.

One aspect of the disclosure is directed to the method of making a multivalent immunogenic composition. In some embodiments, the method comprises providing a host cell comprising (i) a modified genomic DNA of a first virus species comprising a nucleic acid encoding a matrix glycoprotein, wherein the modification creates a replication incompetent virus, comprises a deletion of a gene encoding a surface glycoprotein; and (ii) nucleic acids encoding surface glycoproteins from at least 2 virus species. Then the host cell is infected with a helper virus of the first virus species to produce a population of particles comprising replication incompetent virions comprising the matrix protein of the first virus species and the surface glycoproteins from at least 2 virus species. Finally, the population of particles is isolated. In some embodiments, the helper virus is a vesicular stomatitis virus (VSV).

One embodiment of the disclosure is directed to the method of making a multivalent immunogenic composition comprising transfecting a host cell with a nucleic acid encoding a matrix protein of one virus species and nucleic acids encoding surface glycoproteins of at least two virus species. Then, viral proteins are expressed in the host cell to allow formation of particles comprising surface glycoproteins of at least two virus species. The particles are then purified. Lastly, the multivalent immunogenic composition comprising the purified particles is formulated.

In some embodiments, the method of making a multivalent immunogenic composition is performed where the host cell is selected from bacteria, mammalian cells, insect cells, yeast, and plant cells.

In some embodiments, the present disclosure relates to host cells stably transformed or transfected with the recombinant DNA constructs. The host cell can be prokaryotic (for example, bacterial), lower eukaryotic (for example, yeast or insect) or higher eukaryotic (for example, all mammals, including but not limited to mouse and human). Both prokaryotic and eukaryotic host cells may be used for expression of the desired coding sequences when appropriate control sequences which are compatible with the designated host are used.

Among prokaryotic hosts, E. coli is the most frequently used host cell for expression. General control sequences for prokaryotes include promoters and ribosome binding sites. Transfer vectors compatible with prokaryotic hosts are commonly derived from a plasmid containing genes conferring ampicillin and tetracycline resistance (for example, pBR322) or from the various pUC vectors, which also contain sequences conferring antibiotic resistance. These antibiotic resistance genes may be used to obtain successful transformants by selection on medium containing the appropriate antibiotics. see e.g., Maniatis, Fitsch and Sambrook, Molecular Cloning; A Laboratory Manual (1982) or DNA Cloning, Volumes I and II (D. N. Glover ed. 1985) for general cloning methods. The DNA sequence can be present in the vector operably linked to sequences encoding an IgG molecule, an adjuvant, a carrier, or an agent for aid in purification of Ebola proteins, such as glutathione S-transferase.

In addition, virus gene products can also be expressed in eukaryotic host cells such as yeast cells and mammalian cells. Saccharomyces cerevisiae, Saccharomyces carlsbergensis, and Pichia pastoris are the most commonly used yeast hosts. Control sequences for yeast vectors are known in the art. Mammalian cell lines available as hosts for expression of cloned genes are known in the art and include many immortalized cell lines available from the American Type Culture Collection (ATCC), such as CHO cells, Vero cells, baby hamster kidney (BHK) cells and COS cells, to name a few. Suitable promoters are also known in the art and include viral promoters such as that from SV40, Rous sarcoma virus (RSV), adenovirus (ADV), bovine papilloma virus (BPV), and cytomegalovirus (CMV). Mammalian cells may also require terminator sequences, poly A addition sequences, enhancer sequences which increase expression, or sequences which cause amplification of the gene. These sequences are known in the art.

In some embodiments of the disclosure, the matrix protein of one virus species is selected from a virus in the paramyxovirus, filovirus, flavivirus, orthomyxovirus, arenavirus, retrovirus, coronavirus, matonavirus, togavirus, and alphavirus families. In some embodiments, the matrix protein is from a virus in the paramyxovirus family. In some embodiments, the matrix protein is a Nipah virus matrix protein. In some embodiments, the matrix protein is a Hendra virus matrix protein.

One embodiment of the disclosure relates to a recombinant DNA molecule that includes a vector comprising nucleic acid sequences encoding for viral proteins of interest. The vector can take the form of a plasmid, a eukaryotic expression vector such as pcDNA3.1, pRcCMV2, pZeoSV2, or pCDM8, which are available from Invitrogen, or a virus vector such as baculovirus vectors, retrovirus vectors or adenovirus vectors, alphavirus vectors, and others known in the art. In some embodiments, expression plasmid for NiV M, HeV F and EBOV GP was pCAGGS while NiV F/G and HeV G were in pCDNA3.1.

The genes for viral surface glycoproteins of interest are selected from fusion proteins (F), receptor binding glycoproteins (G), glycoprotein (GP), spike proteins (S), Neuraminidase (NA), amino-terminus glycoprotein (Gn), carboxyl-terminus glycoprotein (Gc), hemagglutinin (H), and P32 protein. In some embodiments of the disclosure, the genes for viral surface glycoproteins of interest are selected from fusion proteins (F), receptor binding glycoproteins (G), and glycoprotein (GP). In some embodiments of the disclosure, the genes for viral surface glycoproteins of interest are selected from spike proteins (S) and neuraminidase (NA). In some embodiments of the disclosure, the genes for viral surface glycoproteins of interest are selected from amino-terminus glycoprotein (Gn), carboxyl-terminus glycoprotein (Gc), hemagglutinin (H), and P32 protein. In some embodiments of the disclosure, the genes for viral surface glycoproteins of interest are multiple spike proteins.

### Administration of Immunogenic Composition

Administration of the immunogenic composition disclosed herein may be carried out by any suitable means, including parenteral injection (such as intraperitoneal, subcutaneous, or intramuscular injection), in ovo injection of birds, orally, or by topical application of the virus (typically carried in a pharmaceutical formulation) to an airway surface. Topical application of the virus to an airway surface can be carried out by intranasal administration (e.g., by use of dropper, swab, or inhaler which deposits a pharmaceutical formulation intranasally). Topical application of the immunogenic composition to an airway surface can also be carried out by inhalation administration, such as by creating respirable particles of a pharmaceutical formulation (including both solid particles and liquid particles) containing the replicon as an aerosol suspension, and then causing the subject to inhale the respirable particles. Methods and apparatus for administering respirable particles of pharmaceutical formulations are well known, and any conventional technique can be employed. Oral administration may be in the form of an ingestable liquid or solid formulation.

When the replicon RNA or DNA is used as a vaccine, the replicon RNA or DNA can be administered directly using techniques such as delivery on gold beads (gene gun), delivery by liposomes, or direct injection, among other methods known to people in the art. Any one or more DNA constructs or replicating RNA described above can be use in any combination effective to elicit an immunogenic response in a subject. Generally, the nucleic acid vaccine administered may be in an amount of about 1-5 ug of nucleic acid per dose and will depend on the subject to be treated, capacity of the subject's immune system to develop the desired immune response, and the degree of protection desired. Precise amounts of the immunogenic composition to be administered may depend on the judgement of the practitioner and may be peculiar to each subject and antigen.

The immunogenic composition may be given in a single dose schedule, or preferably a multiple dose schedule in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. Examples of suitable immunization schedules include: (i) 0, 1 months and 6 months, (ii) 0, 7 days and 1 month, (iii) 0 and 1 month, (iv) 0 and 6 months, or other schedules sufficient to elicit the desired immune responses expected to confer protective immunity, or reduce disease symptoms, or reduce severity of disease.

One embodiment of the disclosure is directed to a method of eliciting an antibody response in a subject. In some embodiments, the method comprises administering an immunogenic composition of any one of claims 16-21 to the subject in an amount sufficient to elicit an antibody response.

In some embodiments, the method of eliciting an antibody response in a subject wherein the subject is a mammal. In some embodiments, the subject is a human. In some embodiments, the subject is a mouse. In some embodiments, the subject is livestock. In some embodiments, the subject is a fowl.

### EXAMPLES

The following examples are presented to illustrate the present disclosure. The examples are not intended to be limiting in any manner.

### Example 1. Multivalent VLPs are Effectively Produced Using HEK 293T Cells

For VLP production, typically HEK 293T cells are infected with codon-optimized mammalian expression plasmids which encode for the expression of a viral protein that drives assembly in and budding from the transfected cells. For example, for the production of multivalent NiV/HeV/EBOV virions, the NiV M and F proteins are able to drive VLP assembly and budding37-39, and Ithinji et al., 2021. For the formation of coronavirus VLPs, the S protein was sufficient to drive assembly and budding40, and VLPs were collected and purified via ultracentrifugation through sucrose 48 hours post transfection.

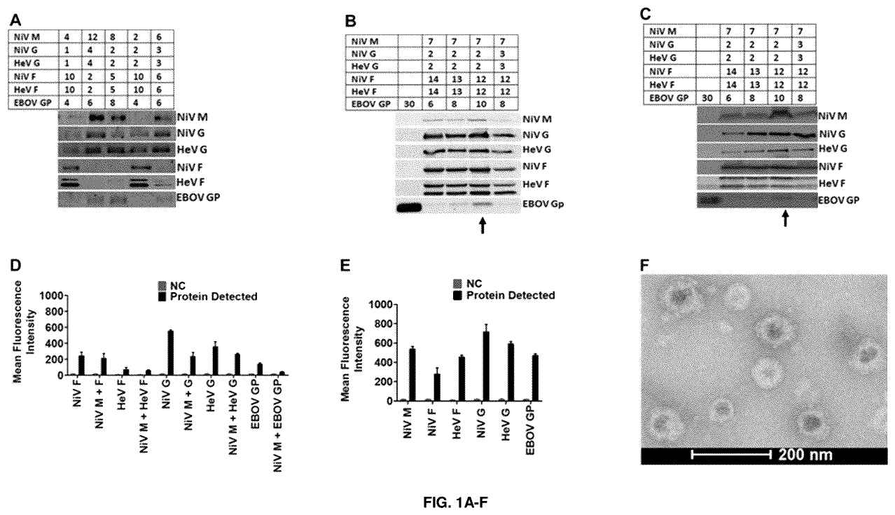

To determine the optimal incorporation of proteins onto VLPs, human embryonic kidney cells (HEK 293T cells) were transfected with different ratios of expression plasmid DNA for NiV M, F, G (Malaysia strain), HeV F, G (original Australian strain), and EBOV GP (Zaire strain), for a total of 30 μg of plasmid DNA per 15-cm dish (FIG. 1A). Incorporation of proteins onto the VLPs was confirmed using standard Western blot analysis. From the initial results of experiments such as that shown in FIG. 1A, The total μg of DNA used was increased to 45 μg/15-cm dish and various DNA ratios tested (FIG. 1B). Transfection of cells with the DNA ratio 7:12:12:2:2:10 μg for NiV M, NiV F, HeV F, NiV G, HeV G and EBOV GP respectively yielded the best protein incorporation into virions (FIG. 1B) and expression of the proteins in cell lysates (FIG. 1C), determined by fluorescent Western blot analysis quantification. Cell surface expression (CSE) levels for each glycoprotein were determined by flow cytometry (FIG. 1D). Incorporation of the proteins onto VLPs was determined by flow virometry (FIG. 1E). The results demonstrated that multiple glycoproteins can be found on the surfaces of viral particles of a single viral preparation. The presence and distribution of the glycoproteins on the surfaces of VLPs was demonstrated by electron microscopy as shown in FIG. 1F. These figures demonstrate that different immunogenic glycoproteins were incorporated onto VLPs following transfection of HEK 293T cells.

### Example 2. Multivalent Pseudotyped VSV Particles were Effectively Produced Using HEK 293T Cells

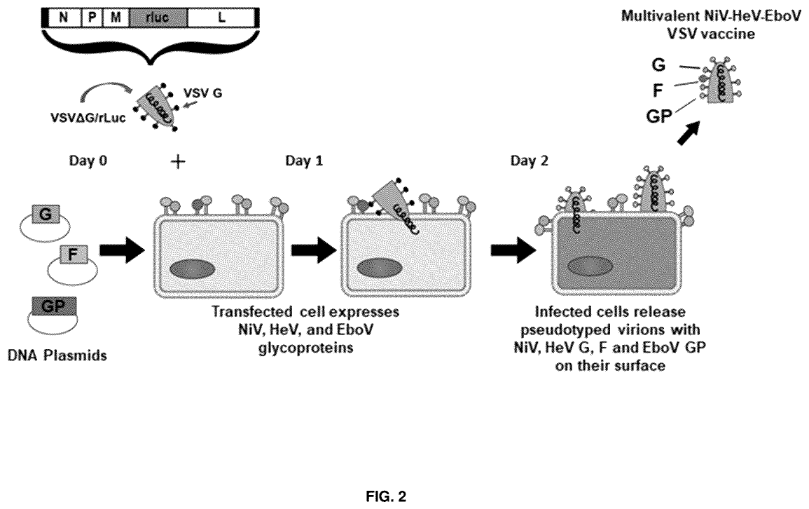

HEK 293T cells were transfected with varying amounts of expression plasmid DNA for NiV F/G, HeV F/G and EBOV GP for 12 hours and then the HEK 293T cells were infected with VSV-rLuc virions as described previously. H. C. Aguilar et al., Journal of Virology, vol. 80, no. 10, 2006, doi: 10.1128/jvi.80.10.4878-4889.2006; and O. A. Negrete, et al., Journal of Virology, vol. 81, no. 19, 2007, doi: 10.1128/jvi.00999-07. FIG. 2 shows a schematic of the multivalent NiV-HeV-EboV VSV particle.

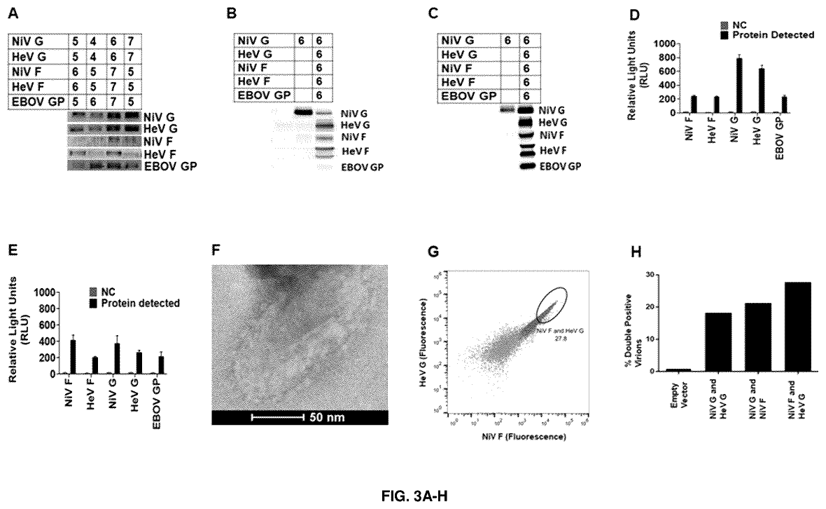

Initial Western blot analysis showed the best pseudotyped VSV protein incorporation would be achieved after transfection with DNA amounts of about 5 to 7 μg for each surface glycoprotein as is seen in FIG. 3A. HEK 293T cells were transfected with 6 μg DNA of the NiV, HeV and EBOV surface proteins for incorporation onto pseudotyped VSV. The proteins were also detected in the cell lysates as verified by Western blot analysis as shown in FIG. 3B. The cells used to produce pseudotyped VSV particles were stained in a similar manner to those used for VLP production. The levels of glycoprotein incorporation were determined by flow virometry, and expression of the glycoproteins on the pseudotyped VSV was also determined by flow cytometry (FIGS. 3D and 3E, respectively). Multiple glycoproteins were found on individual viral particles. FIG. 3F shows a TEM micrograph of a pseudotyped VSV particle showing surface glycoproteins, not observed in negative control samples. The presence of more than one type of glycoprotein on the surface of individual virus particles was demonstrated by flow virometry as is shown in FIG. 3H. FIG. 3G shows an example of the gating of individual viral particles containing both NiV F and HeV G on single particles. The same gating strategy was used to determine the percentage of double-positive virions for various other pairs of glycoproteins that contained extracellular (extraviral) tags as can be seen by FIG. 3H.

### Example 3. Multivalent RVFV-PPRV-GTPV VLPs were Produced Using HEK 293T Cells

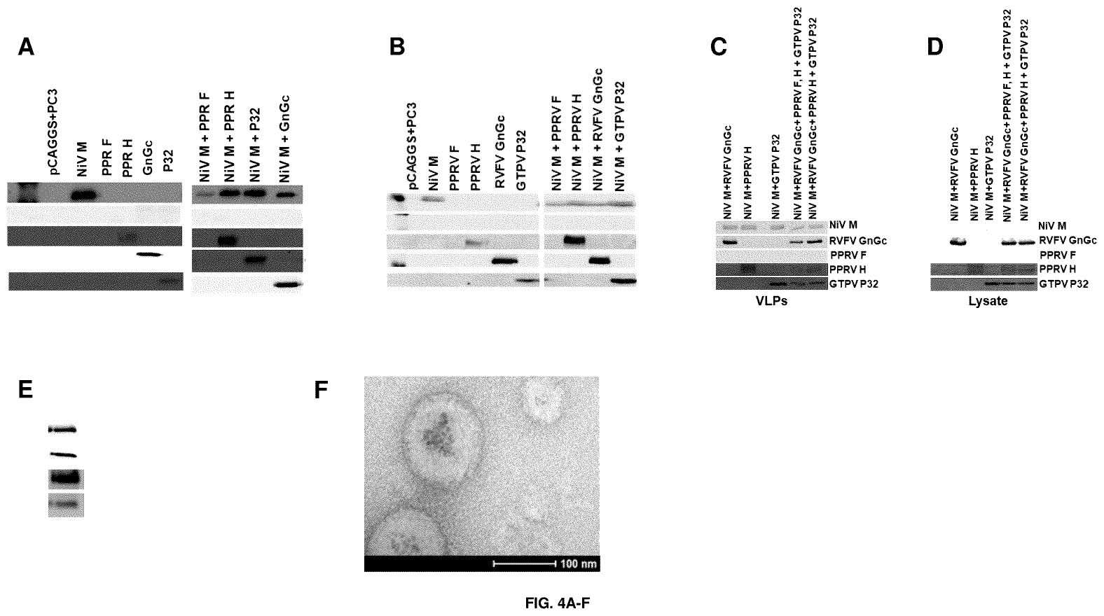

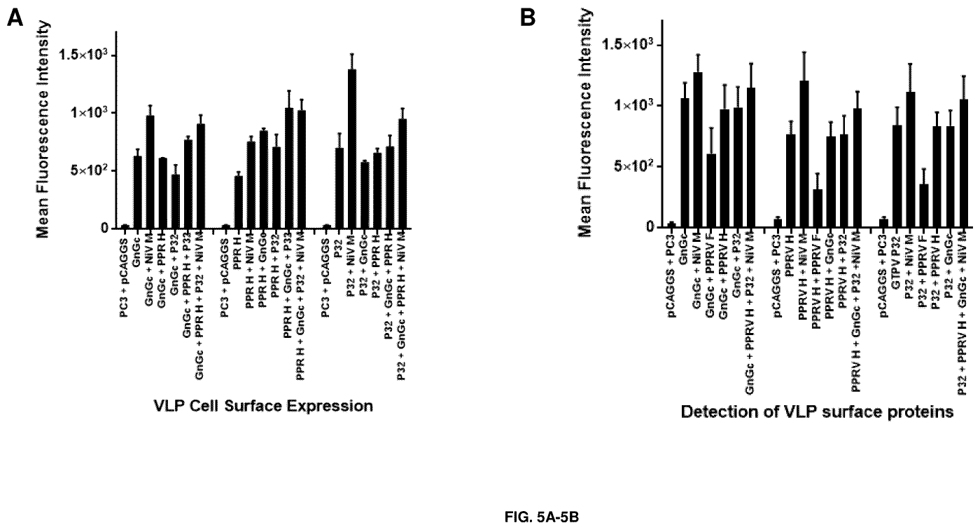

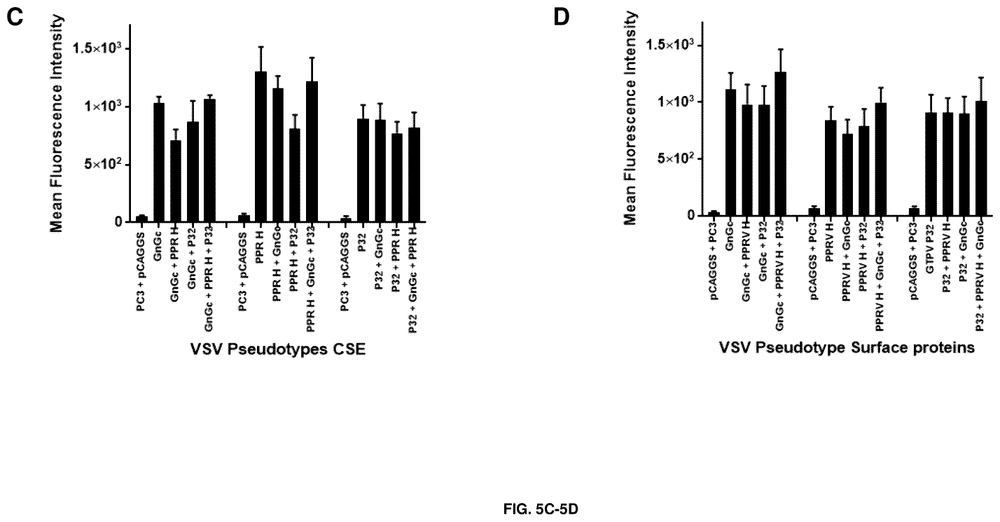

Human embryonic kidney cells (HEK 293T cells) seeded in DMEM were transfected for 24 hours with DNA for NiV M, RVFV GnGc, PPRV F, H and GTPV P32. The cells were transfected with the individual protein DNA at 0.9 μg for NiV and 0.7 μg for the other proteins. Protein incorporation was determined by standard Western blotting as can be seen in FIG. 4A. NiV M was shown to enhance the incorporation of the other proteins as can be seen in FIG. 4B. A reduced incorporation or absence of PPRV F was observed following determination by Western blotting and it was determined that PPRV F reduced the incorporation of the other proteins as can be seen in FIG. 4C. Additionally, cell surface expression and detection of surface proteins can be seen in FIGS. 5A and 5B, respectively. A larger scale 15 cm plate preparation was performed by transfecting 9, 7, 7, 7 μg of DNA for NiV M, RVFV GnGc PPRV H and GTPV P32 respectively, shown in FIG. 4E.

### Example 4. Pseudotyped VSV Particles Effectively Incorporating RVFV-PPRV-GTPV Target Proteins were Produced Using HEK 293T Cells

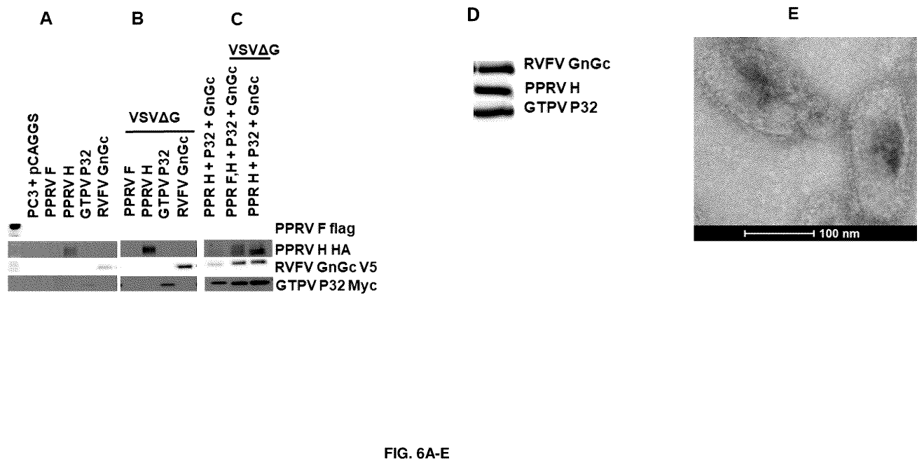

Proteins were incorporated onto the pseudotyped VSV by transfecting human embryonic kidney cells (HEK 293T cells) with DNA for RVFV GnGc, PPRV F and H, and GTPV P32 for 12 hours and then infected them with VSVAG at 1:10000 dilution. Initial transfections were optimized in a 6-well plate with a total of 4 μg per well. Cells were transfected with 1 μg DNA of the individual target proteins without infecting cells with the VSVAG (FIG. 6A). In a different set of wells, the cells were transfected with individual protein DNA and infected the cells with VSVAG after 12 hours and resulting protein levels are shown in FIG. 6B. In a third set of conditions, the cells were transfected with RVFV GnGc, PPRV H and GTPV without infection with VSVAG, followed by transfection with the three proteins plus PPRV F and infection with VSVAG. FIG. 6C shows the protein levels of the third set of conditions. In comparing the Western blots of the three (FIGS. 6A-6C), one can see a marked increase in protein incorporation on the VSV particles when compared to the preparation without VSV. The later experiment was set to determine whether PPRV F was lowering the incorporation of the other proteins onto the pseudotyped VSV which is confirmed. Additionally, cell surface expression and detection of surface proteins can be seen in FIGS. 5C and 5D, respectively. A larger scale 15 cm preparation was performed by transfecting cells with 10 μg of DNA for all the proteins. The proteins were detected using Western immunoblotting as shown in FIG. 6D.

### Example 5: Trehalose Lyophilization Improves Viral Particle Thermostability

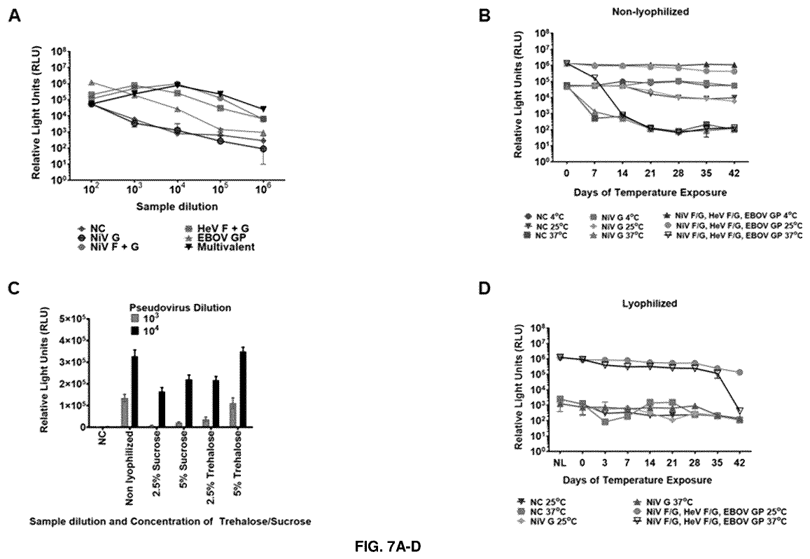

Cell entry capabilities of the monovalent and multivalent VSV virions were used as a surrogate of particle stability to determine the thermostability of the pseudotyped virions. Assessment of viral particle entry was done through a luciferase marker gene. Pseudotyped VSV virions were made incorporating NiV G only (negative control for viral entry), NiV F/G, HeV F/G, EBOV GP only, or all NiV F/G, HeV F/G and EBOV GP (multivalent virions). Bald virions with plasmids pCDNA3.1/pCAGGS served as an additional negative control for viral entry. The pseudotyped virions were used to infect Vero cells at a confluency of 30% and VSV luminescence was recorded 24 hours post-infection (hpi). FIG. 7A shows the cell entry levels of the monovalent and the multivalent virions.

The stability of the pseudotyped virions under different temperature environments were then determined. The multivalent virions were exposed to temperatures of 4° C., 25° C. and 37° C. for a period of up to six weeks and entry levels into Vero cells were determined as shown in FIG. 5A. The virions were stable at 4° C. and 25° C. for the six weeks but lost a log in luminescence detection within a week when exposed to 37° C., and luminescence detection was in the range of the negative controls by the end of the second week as seen in FIG. 7B.

Thermotolerance of virus vaccines has been shown to be enhanced using carbohydrates. A. C. Silva, et al., “Testing a new formulation for Peste des Petits Ruminants vaccine in Ethiopia,” Vaccine, vol. 32, no. 24, 2014, doi: 10.1016/j.vaccine.2014.02.039. The capacities of sucrose and trehalose to preserve the multivalent pseudotyped virions were compared following lyophilization. The virions were lyophilized in 2.5% or 5% sucrose or trehalose for 24 hours at 1:100 and 1:1000 dilutions. Following lyophilization, 5% trehalose preserved the pseudotyped virions completely, yielding luciferase levels similar to those of fresh non-lyophilized virions as seen in FIG. 7C. The multivalent pseudotyped virions were lyophilized using 5% trehalose and exposed to temperatures of 4° C., 25° C. and 37° C. Positive control lyophilized virions were preserved under vacuum at 4° C. for the six-week period, while vials of virions were placed under three different temperatures starting with day 42 vials and ending with day 0 vials (day 42 vials were placed first, then day 35 vials after a week, then days 28, 21, 14 and 7 in that order whereas day 0 represents vials kept under the 4° C. storage conditions). Therefore, all vials were tested on the same day (day 0). The day 0 lyophilized sample shown in FIG. 7D represents values for vials at 4° C. for the entire period. There was minimal loss in viability of the pseudotyped virions following lyophilization when stored at 4° C. or 25° C. Remarkably, a minimal loss in luminescence was observed for the lyophilized virions for up to five weeks at 37° C., a high environmental temperature. Therefore, 5% trehalose lyophilization improved thermotolerance of the pseudotyped VSV virions, allowing the use of this type of vaccine in high temperature climates where maintenance of a cold-chain is impractical.

### Example 6. Multivalent VLPs Elicited Neutralizing Antibody Responses in Hamsters

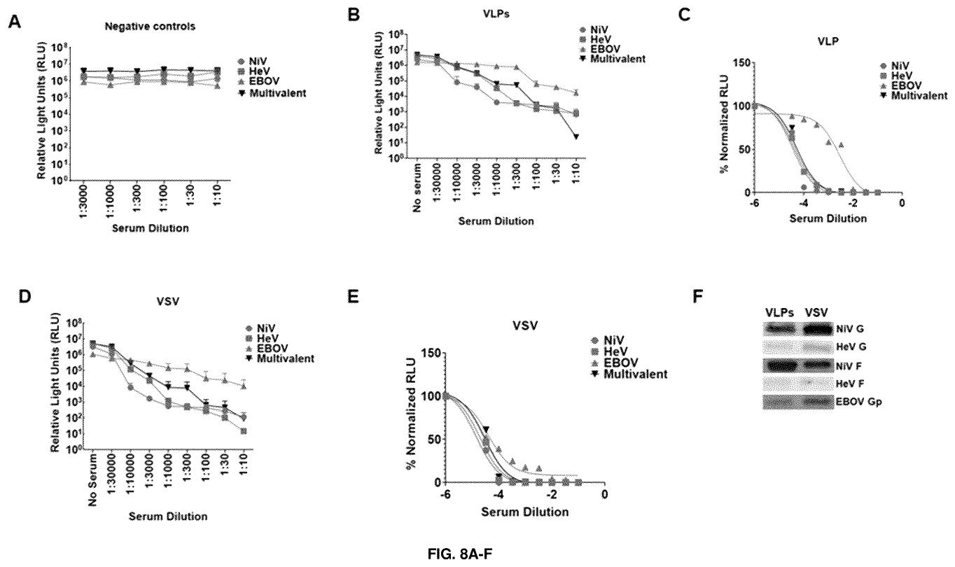

Immunogenic properties of the VLPs in hamsters were determined following vaccination. Five hamsters were vaccinated intramuscularly at six weeks of age using 50 μl (30 μg) of the vaccine preparation in 50 μl of ALUM adjuvant and boosted on days 21 and 42 post first inoculation. Five negative control hamsters were vaccinated with 50 μl of bald VLP preparation in 50 μl of ALUM adjuvant and boosted on days 21 and 42 after first inoculation. The amount of protein inoculated was determined by Bradford assay. Blood samples were collected weekly from day 0 to day 49. Final bleed serum neutralization was determined against fresh batches of pseudotyped VSV virions incorporating NiV F/G only, HeV F/G only, EBOV GP only, or a multivalent VSV virions carrying the three viral glycoproteins using a Renilla Luciferase kit 24 h.p.i. FIG. 8A shows the averaged neutralization for all negative control hamsters, while FIG. 8B shows the averaged neutralization trends for sera from hamsters vaccinated with VLPs. FIG. 8C is a sigmoidal curve derived from the average neutralization for sera from hamsters vaccinated with VLPs. The VLP vaccinated hamsters elicited neutralizing antibodies against all NiV F/G, HeV F/G and EBOV GP, but were weakest against EBOV.

### Example 7. Multivalent Pseudotyped VSV Virions Elicited Strong Neutralizing Antibody Responses in Hamsters

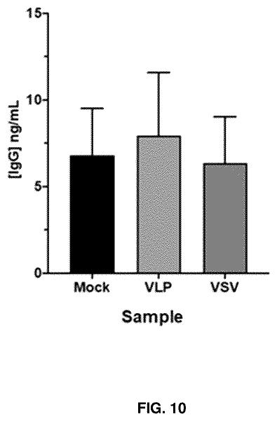

Different groups of hamsters were immunized with the pseudotyped VSV virions. These groups were treated similarly to the group which was immunized with VLPs. Serum samples were also treated as those collected from hamsters vaccinated with the VLPs. FIG. 8D shows the averaged neutralization graphs for antisera derived from hamsters vaccinated with the multivalent pseudotyped VSV vaccine. FIG. 8E is a sigmoid curve derived from the average neutralization read outs for hamsters vaccinated with pseudotyped VSV. The pseudotyped VSV vaccinated hamsters elicited strong neutralizing antibody responses against all NiV F/G, HeV F/G and EBOV GP. Comparison of FIGS. 8C to 8E illustrates that the neutralizing antibody responses to the multivalent VSV vaccine were stronger than those to the multivalent VLP vaccine, likely due to the higher level of incorporation of the glycoproteins into the VSV virion vaccine per equal amount of sample tested, coincidently produced roughly from equal amounts of cells. FIG. 8F shows a difference in the amount of glycoproteins incorporated by VLPs and VSVs as measured by immunoblotting. Because the multivalent pseudotyped VSV preparation had higher incorporation of the glycoproteins but not NiV F and it elicited a stronger antibody response, it was subsequently used for the challenge experiments. The IgG in the sera was quantified for the mock vaccinated hamsters and that from VLPs and pseudotyped VSV vaccinated hamsters using an ELISA. The data shows detection of similar levels of IgG antibodies among all groups indicating that increased neutralization was not due to total antibodies produced, but to specific neutralizing antibodies as is shown in FIG. 10.

### Example 8. Multivalent Pseudotyped VSV Protected Hamsters Against NiV, HeV, or EBOV

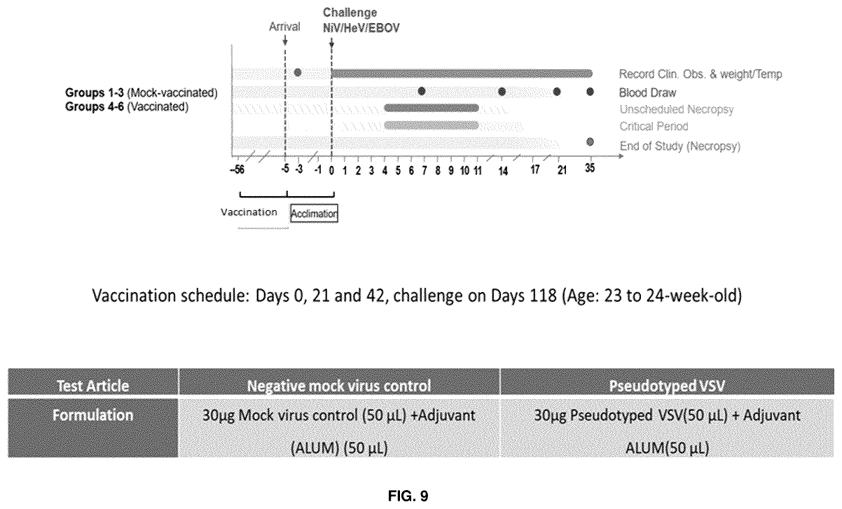

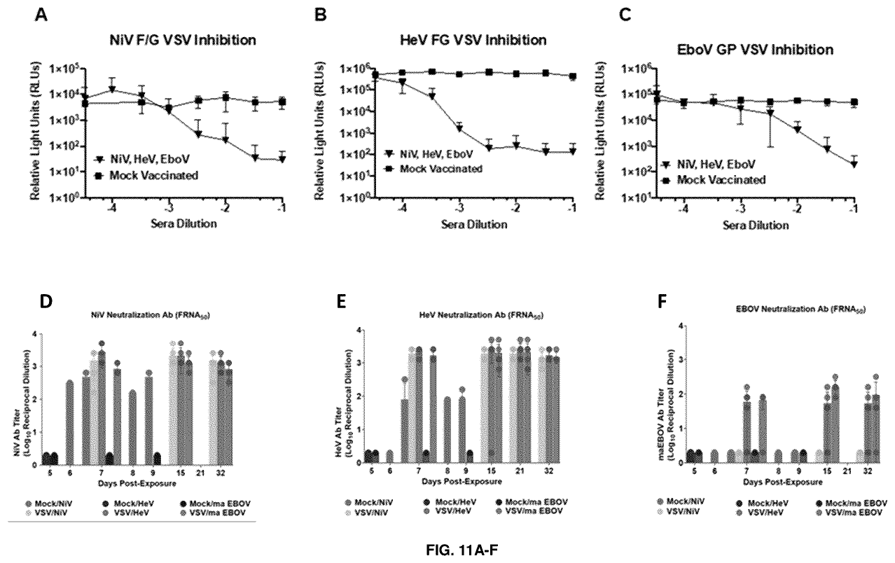

The hamsters were grouped into six groups of six hamsters per group. The first eighteen hamsters were mock vaccinated with phosphate buffered saline (PBS) and Alum, and the second group of eighteen hamsters vaccinated with the multivalent pseudotyped VSV test vaccine. FIG. 9 shows a schematic of the challenge experiment. Sera from the vaccinated hamsters was tested for neutralizing antibodies against NiV, HeV and EBOV prior challenging with live virus. The hamsters vaccinated using the multivalent pseudotyped VSV vaccine elicited a strong antibody response which is shown in FIGS. 11A, 11B and 11C. The sera from the hamsters also neutralized live NiV, HeV and EBOV as seen in FIGS. 11D, 11D, and 11F. In the mock and test vaccine groups, six hamsters were separately challenged with NiV, HeV, or mouse adapted EBOV (maEBOV) five days after arrival at the NIAID Integrated Research BSL-4 laboratory.

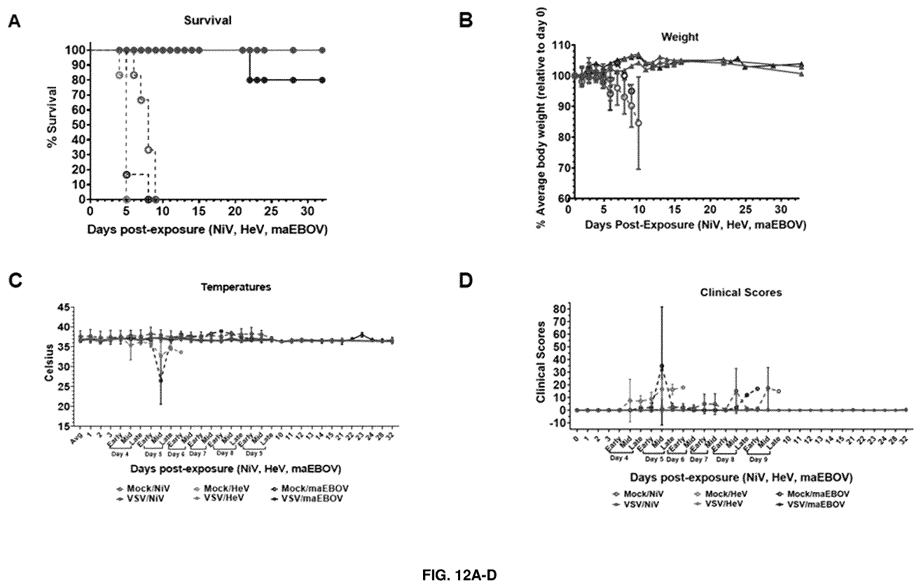

All hamsters in the test vaccine group survived challenge with the three viruses and exhibited no adverse signs of disease as FIG. 12A shows; however, one maEBOV challenged hamster was lost to an unrelated lymphoma. All mock vaccinated hamsters had to be euthanized due to severe disease post-challenge. Mock hamsters challenged with NiV had to be euthanized by day 9, those challenged with HeV by day 5, and those challenged with maEBOV by day 8 post-infection. FIG. 12A shows the survival of the challenged hamsters. A decrease in body weight and temperature for the mock vaccinated group during the course of the experiment was seen as shown in FIGS. 12B and 12C, respectively. During the experiments, the hamsters were also scored clinically based on appearance, respiration, mobility, body temperature, neurological signs, paralysis, seizures and moribund status. The highest score was 15, which was attained if a hamster was unable to access food or water, had a 4° C. drop in body temperature from the baseline and if it was moribund. Other scores fell between 0 and 15. The clinical scores are charted in FIG. 12D. Hamsters were monitored daily during early, mid and late hours. All the hamsters vaccinated with the multivalent VSV vaccine did not show any deviation on any of the parameters from the baseline, while all the mock vaccinated hamsters recorded a significant deviation on a number of these parameters prior to euthanasia. In summary, these challenge experiments demonstrated that the test multivalent VSV vaccine protected hamsters from challenge from virulent NiV, HeV and EBOV infections at 100% efficacy and safety approximately 4 months post-vaccination.

Prior to challenge, Antibody response to NiV, HeV and EBOV was determined prior to challenging with live virus. The pseudotyped VSV vaccinated hamsters elicited neutralizing antibodies against NiV F/G, HeV F/G and EBOV GP, as is seen in FIG. 10.

### Example 9. Multivalent Pseudotyped VSV Vaccinated Hamsters Did not Suffer Pathology Following Challenge with Virulent NiV, HeV and EBOV

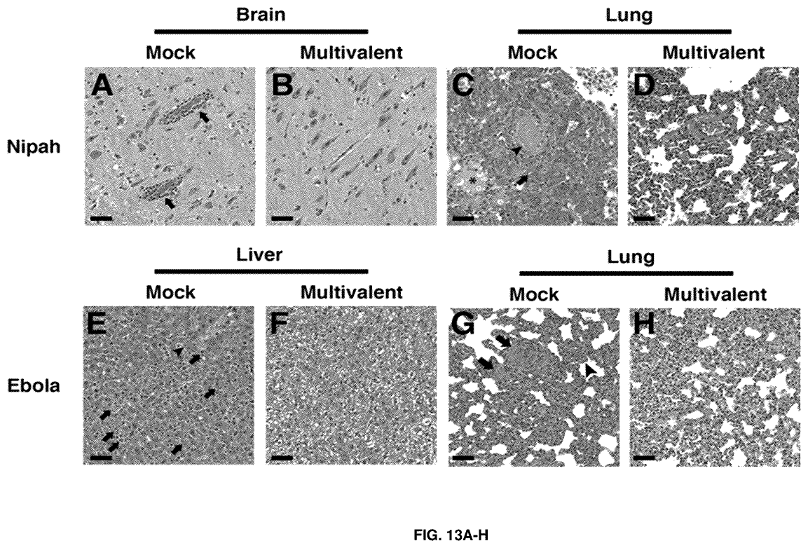

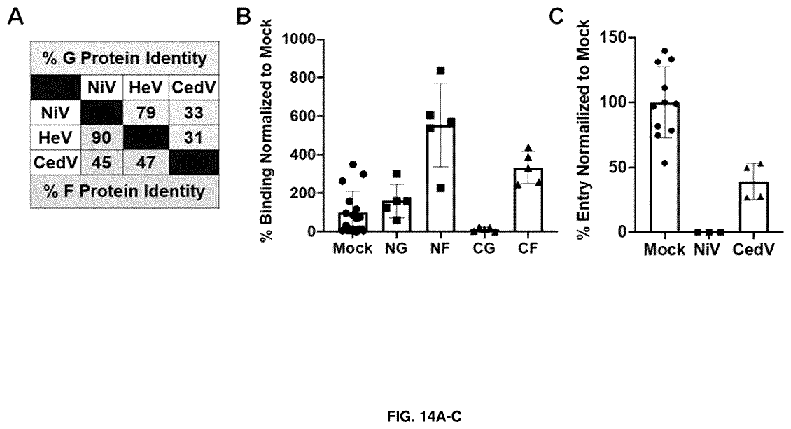

Tissue collection was performed from the challenge hamsters following euthanasia to study histopathology. Analyzed tissues included the brain, liver and lung. FIGS. 13A and 13C demonstrate presence of pathology in the brain and lung respectively for mock vaccinated and NiV challenged hamsters but not the multivalent pseudotyped VSV vaccinated and NiV challenged hamsters. This can be seen in FIGS. 13B and 13D. Pathology due to NiV challenge was similar to that caused by HeV challenge. FIGS. 13E and 13G demonstrate pathology in the liver and lung respectively in the mock vaccinated and EBOV challenged hamsters. The multivalent pseudotyped VSV vaccinated and EBOV challenged hamsters did not suffer pathology as is represented in FIGS. 13F and 13H.

### Example 10. Sera from the Multivalent Pseudotyped VSV Vaccinated Hamsters Cross-Neutralized Pseudotyped VSV Incorporating Glycoproteins for Cedar Virus

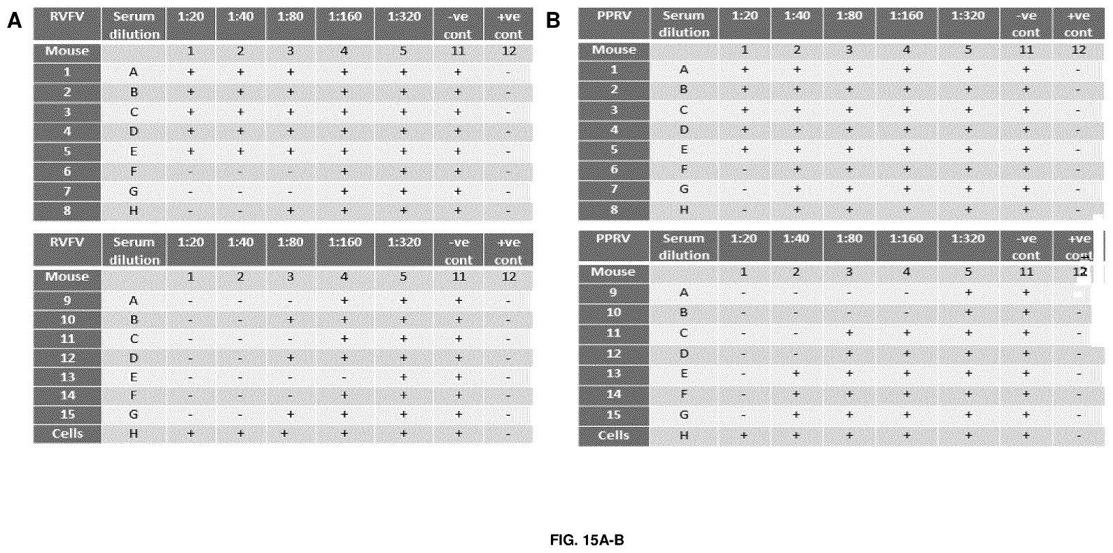

It was then tested whether a virus within the same viral family and containing moderate sequence similarities other family members may also be neutralizable with vaccinated sera. Cedar virus (CedV) has moderate sequence similarities to NiV and HeV G and F as FIG. 14A shows. It was tested to see if CedV may also be neutralizable with the vaccinated sera given the successful neutralization of both NiV and HeV. The binding levels of sera from the vaccinated multivalent VSV hamsters were tested on HEK293T cells expressing the surface G or F glycoproteins from either NiV or CedV. Interestingly, the sera bound relatively well particularly to the F proteins as shown in FIG. 14B. Such results raise the possibility of serum cross-neutralization of other henipaviruses as F protein is relatively more conserved than G protein, shown in FIG. 14A. Therefore, one serum with particularly good F binding levels (serum 6.2) was tested for neutralization of pseudotyped CedV. At a 1:30 dilution of this serum, neutralization of pseudotyped CedV was observed as FIG. 14C shows. Altogether, these results suggest that the multivalent vaccination strategy is capable of yielding cross-protective antibodies against henipaviruses beyond NiV and HeV, justifying further use and optimization of this vaccination strategy towards the goal of broadly protective vaccines.

### Example 11. The Multivalent Pseudotyped VSV Elicited Immune Responses in Mice

Mice were vaccinated intramuscularly on the hind limb at six weeks of age using 50 μl of the vaccine preparation in 50 μl of ALUM adjuvant. Baseline blood samples, weights and body temperatures were all taken before the vaccination. Vaccine boosts were performed on days 21 and 42. Vero cells were infected with 50 μl live RVFV, PPRV and GTPV mixed with 50 μl of serum to test for presence of antibodies in the serum samples. Serum samples from the mice were diluted up to 1:30,000 before mixing. The mix was incubated for one hour at 37° C. in an orbital shaker, dispensed onto 40% confluent Vero cells and incubated for two hours before adding complete media. The Vero cells were incubated at 37° C. and observed daily by microscopy for five days for cytopathic effects (CPE). Residual virus titers were determined using Spearman-Karber method. FIGS. 15A and B show graphs of residual virus titers after neutralizing with different dilutions of sera from the negative control mice. The negative control mice did not elicit antibodies against RVFV, PPRV, or GTPV see column “−ve control”. FIG. 15A and B show graphs of residual virus titers after neutralizing with different dilutions of sera from the pseudotyped VSV vaccinated mice. Mice vaccinated with the multivalent pseudotyped VSV particles elicited antibodies against RVFV, PPRV and GTPV.

### Example 12. Goats Vaccinated with the Multivalent Pseudotyped VSV were Protected from Challenge with Virulent RVFV, PPRV and GTPV

Goats used in these experiments were prescreened for antibodies against RVFV, PPRV and GTPV. 15 goats showing no antibodies against RVFV, PPRV, or GTPV were vaccinated with the multivalent VSV particles while 15 additional goats were vaccinated with a negative control preparation of the VSV without the target proteins. The animals were 6 to 12 months old and were injected intramuscularly on the hind limb using 300 μg of the vaccine preparation in 300 μl of ALUM adjuvant. The vaccine was reconstituted in normal saline and adjuvant added to make 1 ml. Baseline blood samples and body temperatures were taken before the vaccination event. A vaccine boost was given on days 21 and 42. Vero cells were infected with 50 μl live RVFV, PPRV and GTPV mixed with 50 μl of serum to test for presence of antibodies in the serum samples. Serum samples from the goats were diluted up to 1:30,000 before mixing. The mix was then incubated for one hour at 37° C. in an orbital shaker, dispensed onto 40% confluent Vero cells and incubated for two hours before adding complete media. The Vero cells were incubated at 37° C. and observed daily by microscopy for five days for cytopathic effects (CPE). Residual virus titers were determined using the Spearman-Karber method. Goats vaccinated with the multivalent pseudotyped VSV particles elicited antibodies against RVFV, PPRV and GTPV (data not shown). Ten goats, five from the multivalent VSV vaccinated group and five of the negative controls, were separately challenged with RVFV, PPRV, and GTPV on day 49.

### Example 13. Incorporation of Multiple Coronaviral Glycoproteins into VLPs

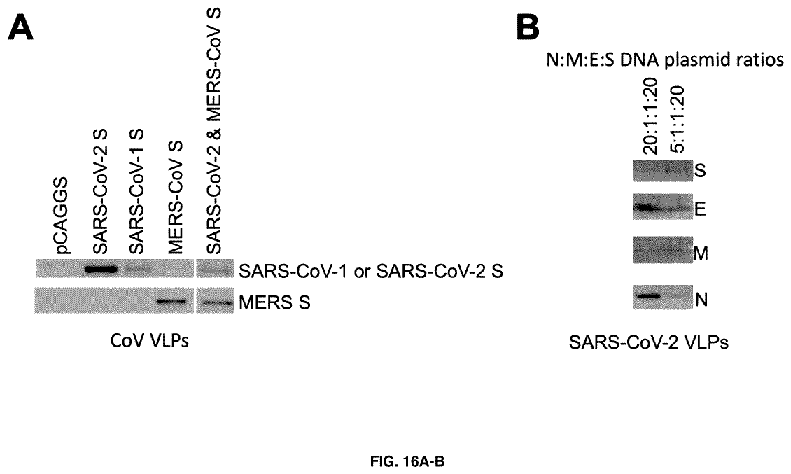

The incorporation of various coronaviral surface proteins into replication-incompetent VLPs or VSV pseudotyped virions was tested. Initial experiments show successful incorporation of individual SARS-CoV-1, SARS-CoV-2, or MERS-CoV S proteins into VLPs, as well as incorporation of two glycoproteins simultaneously into VLPs (SARS-CoV-2 and MERS-CoV S proteins, FIG. 16A); however, further optimization is necessary.

Further, our initial experiment with a couple of DNA ratios showed that it is possible to incorporate various SARS-CoV-2 proteins (S, E, M, and N) onto the same VLPs (FIG. 16B). Further experimentation and flow virometric analysis will be performed to optimize the levels of all these viral proteins into multivalent VLPs, as we have previously performed for other multivalent VLPs.

### Example 14. Incorporation of Multiple Coronaviral S Proteins into Replication Incompetent VSV Pseudotyped Virions

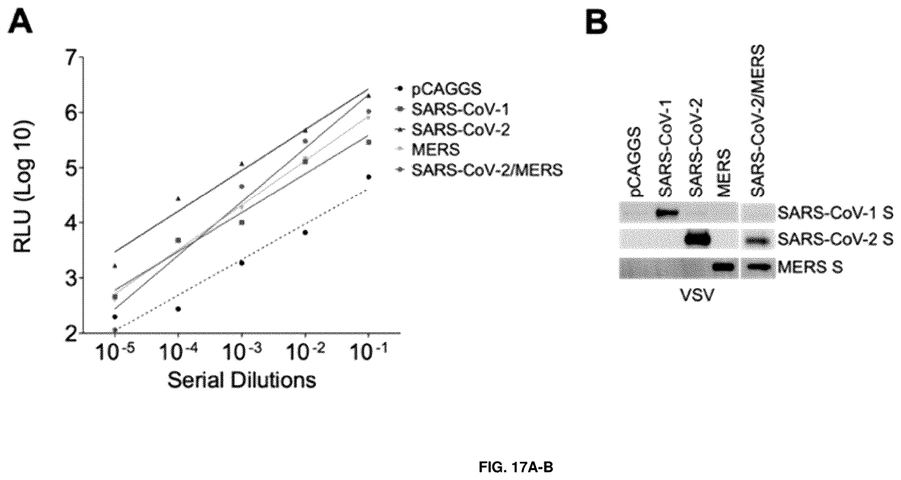

The incorporation of coronaviral glycoproteins into replication incompetent VSV virions was tested. Robust amounts of individual SARSCoV-1, SARS-CoV-2, or MERS into VSV virions were successfully incorporated upon codon-optimization and strategic deletion of large portions of the cytoplasmic tails of the S protein of SARS-CoV-2 (FIG. 17B). Further, using the renilla Luciferase marker gene readout, VSV virions showed 10-100-fold higher signals than the bald VSV background levels, depending on the virus, which is higher than most laboratories have accomplished, at least for SARS-CoV-2 (FIG. 17A). The incorporation of both SARS-CoV-2 and MERS S glycoproteins into VSV virions was tested, and successful incorporation of both S glycoproteins into VSV virions producing a bi-valent SARS-CoV-2/MERS VSV vaccine was accomplished (FIG. 17). Plans are in place to challenge mice with either live SARS-CoV-2 or MERS viruses.

### Example 15. Small Animal Models of Coronavirus Disease

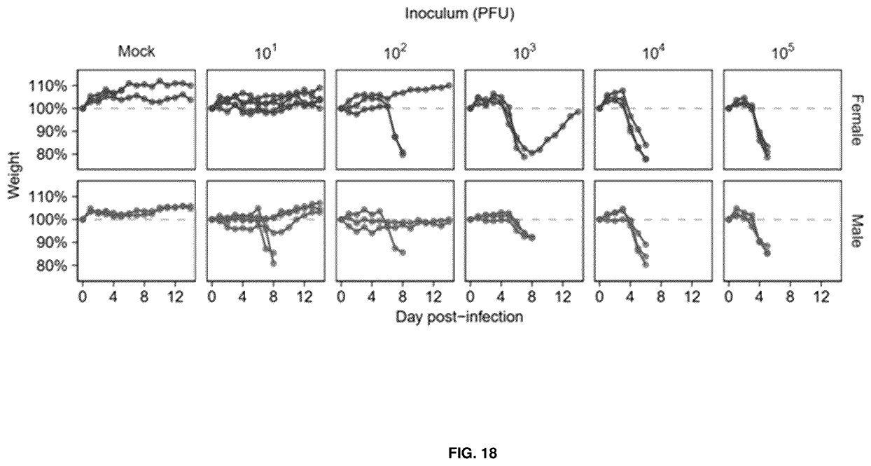

We have previously established several rodent models of disease for the coronaviruses including 1) a C57BL/6 K18-hACE2 transgenic mouse model (The Jackson Laboratory Inc), designated as K18 hACE2 in this application (severe model of disease); a knock-in C57BL/6 model where the human ACE2 gene is driven by the mouse ACE2 promoter, designated as KI hACE2 mice in this application (severe model of disease); a mouse-adapted SARS-CoV-2 strain (obtained under an MTA in collaboration with Ralph Baric at UNC) (mild model of disease). A natural mouse hepatitis virus BSL-2 model (MHV A59 strain, severe model of disease), and a golden Syrian hamster BSL-3 model of SARSCoV-2 disease (mild model of disease). Besides the MHV model, all models are used under animal BSL-3 conditions at Cornell University. The DPP4 mouse model of disease was established for MERS. An example of a viral dose-response experiment showing weight loss for individual K18 hACE2 mice, which yielded mouse mortality (shorter lines), is shown in FIG. 18. Notably, there was a gender difference observed, particularly at the lower viral infection doses, with males having higher levels of severe disease and death as compared to females, which is consistent with human SARS-CoV-2 disease patterns.

### Example 16. Pilot Mouse Experiment Using a Bivalent SARS-CoV-2/MERS S Vaccine

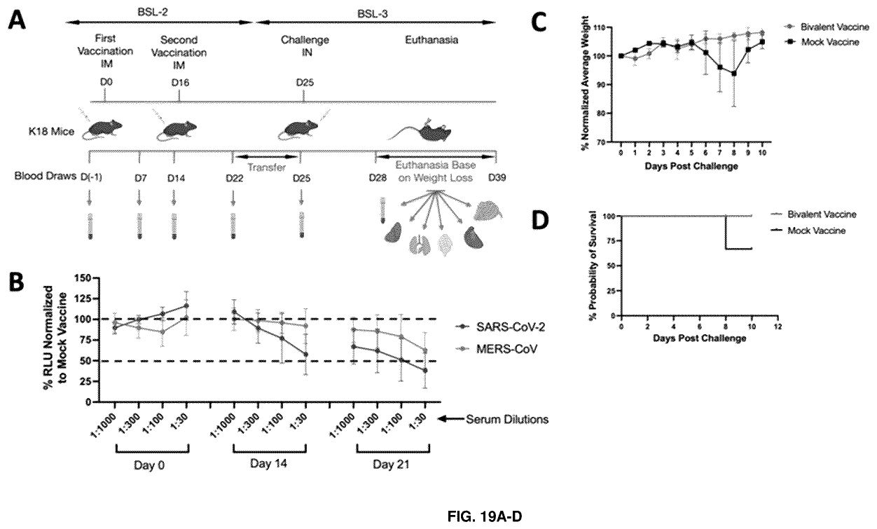

Mice were vaccinated with one or two doses of preliminary non-optimized replication-incompetent bivalent VSV vaccine containing the SARS-CoV-2 and MERS S proteins. A schematic of the vaccinations are shown in FIG. 19A. Sera from mice was collected at days 0, 14, or 21 post-first vaccination (day 21 serum was collected from mice boosted at day 15 post-first vaccination). FIG. 19B shows antibody neutralization results at day 14 and day 21 post-first vaccination. The boosted mice were challenged at day 25 with a dose of 10\hat{ }3 PFU/mouse after seeing promising antibody neutralization results at days 14 and 21. Unfortunately, using this dose did not reach the levels of disease hoped for in the mock-vaccinated animals, in part due to the mice being younger in this experiment (4 weeks old at the time of prime vaccination due to the lack of availability of older mice), as compared to the 8-week old mice challenged previously in FIG. 18. However, even with this caveat, the results indicated that while 2 of the 3 mice in the mock-vaccinated control group had severe viral infection (one death and the other one ˜10% weight loss), the group of 3 vaccinated mice all were protected against all signs of viral disease, with no weight loss and only a normal pattern weight gain observed, and no clinical signs or mortality observed. A higher dose of virus could have been used to challenge and achieve 100% mortality (10\hat{ }4-10\hat{ }5 PFU/mouse), and plans are in place to do so in the future, but this pilot experiment shows promising data to support the subsequent studies. Importantly, the sera form the immunized mice prior to challenge successfully neutralized both SARS-CoV-2 and MERS-CoV pseudotyped virions.

### Example 17. Bivalent VSV Pseudotyped Vaccine Induced Neutralizing Antibodies Against Pseudotyped SARS-CoV-2 Virus and H1N1-PR8 Virus in Mice

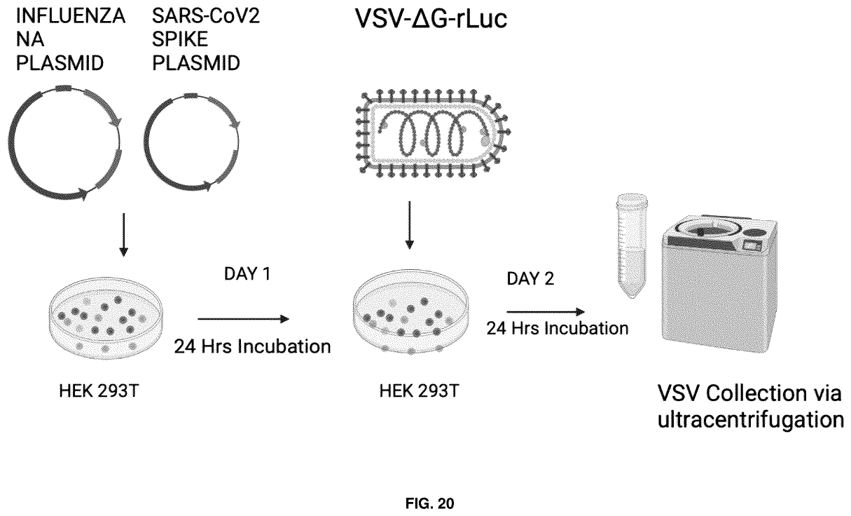

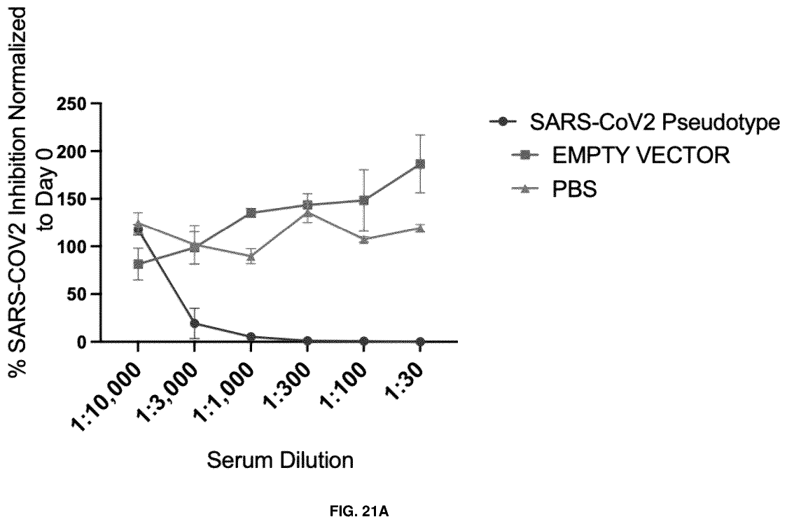

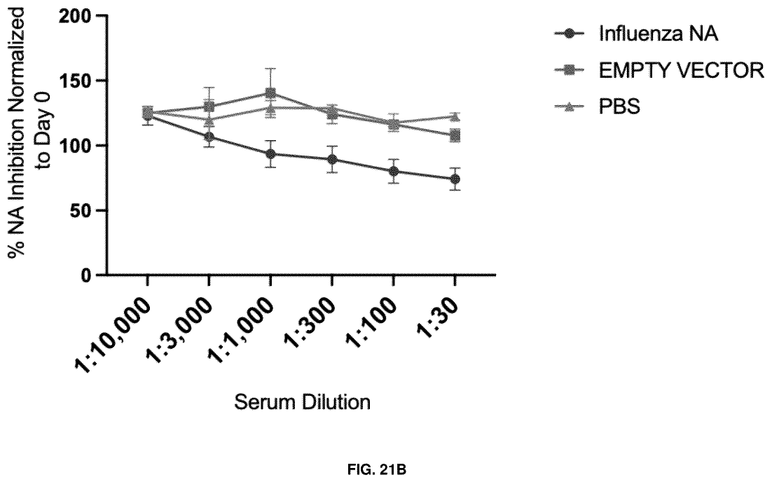

Surface glycoproteins were incorporated onto the pseudotyped VSV platform by transfecting human embryonic kidney cells (HEK 293T cells) with DNA for SARS-CoV2 Spike protein and Influenza neuraminidase (NA) for 24 hours and then infected them with VSVAG for 24 hours. FIG. 20 shows a schematic of the surface glycoprotein incorporation onto the pseudotyped VSV platform. FIG. 21A shows a graph representing the serum dilution and the percent SARS-CoV-2 inhibition. FIG. 21B shows a graph representation of serum dilution and the percent influenza NA inhibition. The Bivalent VSV pseudotyped vaccine induces neutralizing antibodies against pseudotyped SARS-CoV-2 virus and H1N1-PR8 virus in mice.

### Example 18. Comparison of Multivalent Replication Incompetent VLP and Pseudotyped VSV in Development of Broadly Protective and Effective Anti-Coronavirus Vaccines

Based on the success of the preliminary multivalent NiV/HeV/EBOV vaccine pre-clinical experiments showing 100% efficacy and 100% safety were obtained, the incorporation of multiple spike (S) glycoproteins of SARS-CoV-1, SARS-CoV-2, and/or MERS coronaviruses into multivalent VLP (FIG. 16) or pseudotyped VSV particles (FIG. 17) was tested. Although further optimization will be required, preliminary data show promise on 1) the incorporation of multiple SARS-CoV-1, SARS-CoV-2, and MERS-CoV proteins into viral particles; 2) the viral entry levels over background of those particles measured via luciferase activity as seen in FIGS. 16 and 17, and 3) the ability of the initial non-optimized bivalent SARS-CoV-2/MERS-CoV S VSV preparation to induce neutralizing antibodies against both CoVs (FIG. 19).

### General Methodologies Used Throughout.

### Cell Cultures

The cells used in this study were sourced from ATCC and used below their 20th passage.

Cells were tested to be mycoplasma free. The human embryonic kidney 293Tcells and Vero cells were grown in Dulbecco's minimum essential medium supplemented with 10% fetal bovine serum (FBS) and 1% penicillin/streptomycin. The cells were maintained in this medium throughout the study.

### Cell Transfections

Human embryonic kidney (HEK) 293T cells were grown in Dulbecco's complete medium to a 80-90% confluent cell monolayers. The cells were transfected with the plasmid constructs for 8 hours using Lipofectamine 2000 (Invitrogen Inc) according to the manufacturer's guidelines. After 48 hours post-transfection, the VLP-containing cell supernatants (SUP) were harvested for concentration and purification of the VLPs. For the pseudotyped VSV preparations, the cells were infected with 1:10000 dilution of VSVAG after 12-14 hours following transfection. The pseudotyped VSV's and VLPs' SUPs were harvested at the same time. The cells were lysed using RIPA buffer to determine protein content in the lysates and also stained with primary and secondary antibodies to determine protein cell surface expression via flow cytometry.

### VLPs Harvesting and Purification

VLPs and pseudotyped VSV released in the transfected cell supernatants were harvested and clarified by centrifugation at 2200 rpm for 10 minutes at 4° C. The clarified SUPs were concentrated by ultracentrifugation through 20% sucrose cushion in TN buffer (0.1 M NaCl; 0.05 M Tris-HCL, pH 7.4) at 110,000 g for 1.5 hours at 4° C. The resulting VLP and pseudotyped VSV pellet was resuspended in endotoxin-free 5% sucrose buffer and stored at 4° C. for short term use or −80° C. for extended storage. 293T cells were also transfected with empty pCDNA3.1 and pCAGGS plasmids and their supernatants similarly processed to be used as negative controls.

### Protein Determination

The total protein concentration of the purified the VLPs and pseudotyped VSV preparations was measured using the BCA (Bicinchoninic acid) method following the manufacturer's instructions (Thermo Scientific Laboratories).

### Western Blotting

The collected VLPs and pseudotyped VSV virions were analyzed by Western blotting to determine incorporation of glycoproteins. 10 μl of 6×SDS-PAGE dye were added to each sample, individually loaded onto each lane of a polyacrylamide gel and ran at 100V for 2 hours. The proteins were transferred onto a nitrocellulose membrane at 0.5 A for 1.5 hours. Glycoproteins NiV M and NiV F were blotted in 1:500 dilution of mouse anti-flag, EBOV GP in 1:500 mouse anti-V5, while NiV G, HeV F and HeVG were blotted in 1:1000 dilution of rabbit anti-Myc, anti-AU1 and anti-HA primary antibodies respectively. Alexa Fluor 647 goat anti-mouse IgG and Alexa Fluor 488 goat anti-rabbit IgG (Life Technologies) fluorescent secondary antibodies were used at a 1:1000 dilution.

### Flow Virometry and Cytometry

VLP and pseudotyped VSV samples were stained with primary mouse anti-flag, anti-V5 and rabbit anti-MyC, anti-AU1, and anti-HA antibodies at a dilution of 1:200 dilution for 1 h at 4° C. The samples were then washed with FACs buffer (phosphate-buffered saline with 1% fetal bovine serum) by ultracentrifuging twice at 110,000 g for 1 h at 4° C. Goat anti-rabbit 647 and goat anti-mouse 488 fluorescent secondary antibodies (Invitrogen®) were then added also at 1:200 dilution and allowed to bind at 4° C. for 30 minutes. The samples were washed once in FACs buffer by spinning at 110,000 g and resuspended in PBS with 0.5% paraformaldehyde (PFA). The surface proteins were detected using a Guava easyCyte 8HT flow cytometer in which the forward and side scatter (FSC and SSC) settings were slightly modified to detect smaller particles. The relatively small viral particles were differentiated from suspension buffer debris by gating in the forward versus side scatter plot. The transfected 293T cells were collected and stained in a similar manner as VLPs, but were spun 3 times at each stage at 2200 rpm in a 96 well plate. They were also resuspended in PBS with 0.5% PFA and the protein cell surface expression (CSE) determined using the Guava easyCyte 8HT flow cytometer.

### Viral Infectivity Assay

Vero cells at 40% confluency were infected with the pseudotyped VSV at 1:100 to 1:1,000,000 dilutions and incubated for 24 hours. The cells were lysed and mean luminescence was taken using Renilla Luciferase assay as per manufacturer's instructions (Promega®).

### Lyophilization Protocol

Negative plasmid pseudotyped VSV and NiV G only pseudotyped VSV served as the negative controls. The controls and the multivalent VSV were serially diluted to 1:100, 1:1000, 1:10000, 1:100000 and 1:1000000 in 5% trehalose. Tubes containing the diluted samples were immersed in liquid nitrogen for 1 minute and transferred onto lyophilizing jars. Lyophilizing was run for 24 hours. The lyophilized pseudotyped virus was stored at 4° C. in a vacuum jar until required.

### Electron Microscopy

VLPs and the pseudotyped VSV were collected and purified as previously described. The virions were adsorbed on a Formvar carbon coated copper grid by floating it on a drop of sample suspension for 15 minutes and then fixing using 2% formaldehyde/2% glutaraldehyde solution in 0.1M cacodylate buffer. The grids were blotted, and then negatively stained with 1% aqueous uranyl acetate and viewed using a FEI T20 electron microscope.

### Protocol for Immunizing Hamsters

Animal studies were approved by the Institutional Animal Care and Use Committee (IACUC) (Cornell Protocol number 2018-0063). Fifteen five weeks old female hamsters (Charles River Laboratories) were housed in cages for two weeks in the East Campus Research Facility (ECRF), Cornell University, before starting the immunization protocol experiments. Five hamsters per treatment were immunized intramuscularly with 50 μl of the test sample in 50 μl of ALUM as follows; group one was injected with the empty plasmid controls, group two with the VLPs and group three with the pseudotyped VSV. The total protein in the test vaccines was 30 μg determined using the BCA method. The hamsters were given a vaccine boost on days 21 and 42. They were bled on days 0, 7, 14, 21, 28, 35, 42, and terminated on day 49 and euthanized.

### Serum Neutralization

Serum samples from both the VLP and pseudotyped VSV vaccinated hamsters were diluted 1:10, 1:30, 1:100, 1:300, 1:1000, 1:3000, 1:10000, and 1:30,000. NiV F/G pseudotyped VSV was diluted 1:10000, the HeV F/G pseudotyped virus 1:1000, the EBOV GP pseudotyped VSV 1:100 and the multivalent pseudotyped VSV 1:10000 as previously determined. Each pseudotyped VSV was dispensed onto microcentrifuge tubes and equal amounts of each serum dilution added. They were incubated for one hour at 37° C. in a shaker and then 100 μl dispensed onto Vero cells at 40% confluency in duplicates. Neutralization of the different pseudotyped VSV was measured using the viral infectivity assay.

### Antibody Binding Assay

10 cm plates of HEK293T cells were transfected with 15 ug of NiV G, NiV F, CedV G, or CedV F or negative control pCAGGS vector expression plasmids in the presence of PEI (1 mg/mL) at a 4:1 transfection to plasmid ratio. After 24 hours, cells were incubated with hamster serum for 30 minutes on ice. Serum samples from Mock or VSV vaccinated hamsters were diluted 1:30 in PBS with 1% BCS blocker prior to incubation with cells. Cells were washed 3× (300×g, 5 minutes, 4 C) with cold PBS. Goat anti-hamster secondary antibody (1:5,000) was added to the cells and left to bind for 30 minutes on ice prior to washing and detection by flow cytometry.

### Protocol for Challenge Experiments

A new batch of 36 hamsters was housed at the Cornell University ECRF. Eighteen hamsters were vaccinated intramuscularly with 50 μl (30 μg) of the multivalent pseudotyped VSV in 50 μl of ALUM. Eighteen hamsters in the negative control group were given 50 μl (30 μg) of mock virus in 50 μl of ALUM. Hamsters were at ECRF for 113 days before transfer to National Institute of Allergy and Infectious Diseases (NIAID) Integrated Research Facility (IRF). At the IRF, the hamsters were allowed to acclimatize for 5 days before onset of challenge experiments. Challenge experiments were done under animal protocol number IRF-022E. The mock and test vaccine hamsters were separated into six treatment groups of six hamsters per group and challenge virus was introduced intraperitoneal. In the mock and pseudotyped VSV vaccinated clusters, six hamsters were challenged with Malysian strain NiV (19,680 PFU), six with HeV (10,020 PFU) and six with mouse adapted EBOV-maEBOV (10,900 PFU). The approximate LD50 for the challenge viruses was 3280 pfu for NiV, 850 pfu for HeV, and above 110 pfu for ma-EBOV. The higher challenge doses were considered based on the age of hamsters. The study period was 35 days.

### Histopathology

Lung, brain, and liver tissue samples were fixed with 4% paraformaldehyde, paraffin embedded and cut into 3.5-μm sections. The tissue sections were stained with H&E.

### Serum Neutralization

Serum samples from both the VLP and pseudotyped VSV vaccinated hamsters were diluted 1:10, 1:30, 1:100, 1:300, 1:1000, 1:3000, 1:10000, and 1:30,000. NiV F/G pseudotyped VSV was diluted 1:10000, the HeV F/G pseudotyped virus 1:1000, the EBOV GP pseudotyped VSV 1:100 and the multivalent pseudotyped VSV 1:10000 as previously determined. Each pseudotyped VSV was dispensed onto microcentrifuge tubes and equal amounts of each serum dilution added. They were incubated for one hour at 37° C. in a shaker and then 100 μl dispensed onto Vero cells at 40% confluency in duplicates. Neutralization of the different pseudotyped VSV was measured using the viral infectivity assay.

### Viral Infectivity Assay

Vero cells at 40% confluency were infected with the various pseudotyped VSV at 1:100 to 1:1000000 dilutions and incubated for 24 hours. The cells were lysed and mean luminescence was taken using Renilla Luciferase assay as per manufacturer's instructions (Promega®).

### Fluorescence Neutralization Assay 50 (FRNA50)

All assays were run on irradiated and heat-inactivated sera. Irradiation and heat-inactivation were performed as described in the “Serology” methods section. VeroE6 (BEI #NR596) cells were seeded at 3×104 in 100 μL DMEM+10% FBS in 96 well Operetta plates (Greiner Bio-One). The following day, a series of twelve-point dilutions, each 1:2, was performed in duplicates (1:20, 1:40, 1:60, etc.) in 96 well 1.2 mL cluster tubes (Corning). Starting dilution depended on the virus, for Hendra virus (HeV) and Nipah virus (NiV), starting dilution was 1:40, for mouse adapted Ebola virus (maEBOV), the starting dilution was 1:20. Then, stock Hendra virus, Nipah virus and mouse adapted Ebola virus was diluted in serum free media and was added to the sera in each cluster tube at 0.5 multiplicity of infection (MOI) for HeV, 1.0 MOI for maEBOV 0.1 MOI for NiV using a liquidator, doubling the total volume in each well and further diluting sera 1:2. Thus, the final starting dilution was 1:80 and 1:40. The sera/virus mixture was then mixed by pipetting up and down with the liquidator and incubated for 1 hour 37° C./5% CO2. Assay was performed in accordance to the methods described in “Scalable, semi-automated fluorescence reduction neutralization assay for qualitative assessment for Ebola virus-neutralizing antibodies in human clinical samples”. After the sera/virus mixtures was added to the plates, plates were incubated for 24 hours. For the fluorescence staining, the primary antibody was HeV Ab Mix-PA8903&8904 Termination (IBT) prepared at 1:2000, Mouse antibody, EBOV VP40 BMD04B007 All (USAMRIID) prepared at 1:2000 and Rabbit Ab NIV PA8905 Terminal (ThermoFisher) at 1:2000 in blocking buffer at room temperature. Plates were incubated with primary antibody for 60 minutes on a rocker. The secondary antibody was Goat α-rabbit IgG (H+L), Alexa Fluor 594 Conjugate (Life Technologies) prepared at 1:2500 in 1×PBS. Plates were incubated with secondary antibody at room temperature for 30 minutes on a rocker and in the dark. The fluorescence intensity of a sample at each dilution was compared to the FRNA50 values, and the lowest dilution that is equal to or less than the FRNA50 value was recorded.

### Antibody Binding Assay

10 cm plates of HEK293T cells were transfected with 15 ug of NiV-G, NiV-F, CedV-G, or CedV-F plasmid with PEI (1 mg/mL) at a 4:1 transfection to plasmid ratio. After 24 hours, cells were incubated with hamster serum for 30 minutes on ice. Serum samples from Mock or VSV vaccinated hamsters were diluted 1:30 in PBS with 1% BCS prior to incubation. Cells were washed 3× (300×g, 5 minutes, 4 C) with cold PBS. Goat anti-hamster secondary antibody (1:5,000) was added to the cells and left to bind for 30 minutes on ice. Signals were detected by flow cytometry.

What is claimed is:

1. A composition comprising a population of particles that are replication-incompetent virions or virus-like particles, wherein the particles each comprise
(i) a matrix glycoprotein base, wherein the matrix glycoprotein is from a first virus species, and
(ii) surface glycoproteins from at least two virus species.

2. The composition of claim 1, wherein the at least two virus species include a virus species different from the first virus species.

3. The composition of claim 1 or 2, wherein the at least two virus species include virus species from two virus families.

4. The composition of claim 3, wherein the virus families are selected from filoviruses, rhabdoviruses, coronaviruses, arenaviruses, orthomyxoviruses, retroviruses, flaviviruses, togaviruses, alphaviruses, and paramyxoviruses.

5. The composition according to any one of claims 1-4, wherein the first virus species is selected from Nipah (NiV), Hendra (HeV), Ebola (EBOV), Rift valley fever (RVFV), peste des petits ruminants (PPRV), goat pox (GTPV), influenza viral species, and beta-coronaviral species.

6. The composition of claim 5, wherein the beta-coronaviral species are SARS-CoV-1, SARS-CoV-2, MERS-CoV, and their variants.

7. The composition of claim 1, wherein the surface glycoproteins are selected from NiV-F, NiV-G, HeV-F, HeV-G, and EBOV-GP.

8. The composition of claim 1, wherein the surface glycoproteins are selected from SARS-CoV-1 spike protein, SARS-CoV-2 spike protein, and MERS-CoV spike protein.

9. The composition of claim 1, wherein the surface glycoproteins are selected from RVFV GnGc, PPRV F and H, and GTPV P32.

10. The composition of claim 1, wherein the surface glycoproteins are selected from SARS-CoV-2 spike protein and influenza neuraminidase (NA).

11. The composition of claim 1, wherein the population of particles is a replication-incompetent virion.

12. The composition of claim 11, wherein the virion is a pseudotyped vesicular stomatitis virus (VSV).

13. The composition of claim 12, wherein the pseudotyped VSV is a recombinant VSV wherein the native envelope G protein is removed.

14. The composition of claim 13, wherein the recombinant VSV native G protein is replaced by a reporter protein.

15. The composition of any one of claims 11-14, wherein the virion comprises surface glycoproteins from two virus species.

16. The composition of any one of claims 11-14, wherein the virion comprises surface glycoproteins from three virus species.

17. The composition of claim 15 or 16, wherein the virion comprises surface glycoproteins from virus species of at least two virus families.

18. The composition of any one of claims 11-17, wherein the surface glycoproteins are from virus families selected from filoviruses, rhabdoviruses, coronaviruses, arenaviruses, orthomyxoviruses, retroviruses, flaviviruses, togaviruses, alphaviruses, and paramyxoviruses.

19. The composition of any one of claims 11-18, wherein the surface glycoproteins are from viruses selected from Nipah (NiV), Hendra (HeV), Ebola (EBOV), Rift valley fever (RVFV), peste des petits ruminants (PPRV), goat pox (GTPV), influenza viral species, and beta-coronaviral species.

20. The composition of claim 18, wherein the beta-coronaviral species are SARS-CoV-1, SARS-CoV-2, MERS-CoV, and their variants.

21. The composition of claim 18 or 19, wherein the surface glycoproteins are selected from NiV-F, NiV-G, HeV-F, HeV-G, and EBOV-GP.

22. The composition of claim 19 or 20, wherein the surface glycoproteins are selected from SARS-CoV-1 spike protein, SARS-CoV-2 spike protein, and MERS-CoV spike protein.

23. The composition of claim 19, wherein the surface glycoproteins are selected from RVFV GnGc, PPRV F and H, and GTPV P32.

24. The composition of claim 19, wherein the surface glycoproteins are selected from SARS-CoV-2 spike protein and influenza neuraminidase (NA).

25. The composition of claim 1, wherein the population of particles comprises a virus-like particle (VLP).

26. The composition of claim 25, wherein the VLP comprises a matrix protein selected from a virus in one of the following families: paramyxovirus, filovirus, flavivirus, orthomyxovirus, arenavirus, retrovirus, coronavirus, rhabdoviruses, togavirus, and alphavirus.

27. The composition of claim 26, wherein the matrix protein is from a virus in the paramyxovirus family.

28. The composition of claim 26, wherein the matrix protein is a Nipah virus matrix protein.

29. The composition of claim 26, wherein the matrix protein is a Hendra virus matrix protein.

30. The composition of any one of claim 25 or 28, wherein the VLP comprises surface glycoproteins from two virus species.

31. The composition of any one of claim 25 or 28, wherein the VLP comprises surface glycoproteins from three virus species.

32. The composition of claim 30 or 31, wherein the virion comprises surface glycoproteins from virus species of at least two virus families.

33. The composition of any one of claims 25-32, wherein the surface glycoproteins are from viruses selected from the virus families of filoviruses, rhabdoviruses, coronaviruses, arenaviruses, orthomyxoviruses, retroviruses, flaviviruses, togaviruses, alphaviruses, and paramyxoviruses.

34. The composition of any one of claims 25-33, wherein the surface glycoproteins are from viruses selected from two or more of the following Nipah (NiV), Hendra (HeV), Ebola (EBOV), Rift valley fever (RVFV), peste des petits ruminants (PPRV), goat pox (GTPV), influenza viral species, and beta-coronaviral species.

35. The composition of claim 34, wherein the beta-coronaviral species are SARS-CoV-1, SARS-CoV-2, MERS-CoV, and their variants.

36. The composition of any one of claims 25-34, wherein the surface glycoproteins are selected from NiV-F, NiV-G, HeV-F, HeV-G, and EBOV-GP.

37. The composition of any one of claims 25-34, wherein the surface glycoproteins are selected from SARS-CoV-1 spike protein, SARS-CoV-2 spike protein, and MERS-CoV spike protein.

38. The composition of any one of claims 25-34, wherein the surface glycoproteins are selected from RVFV GnGc, PPRV F and H, and GTPV P32.

39. The composition of any one of claims 25-34, wherein the surface glycoproteins are selected from SARS-CoV-2 spike protein and influenza neuraminidase (NA).

40. An immunogenic composition comprising the composition of any one of claims 1-39 and a pharmaceutically acceptable carrier.

41. The composition of claim 40 further comprising a carbohydrate.

42. The composition of claim 41, wherein the carbohydrate is at a range of concentration between about 1 wt % and 10 wt %.

43. The composition of either claim 41 or 42, wherein the carbohydrate is at a concentration of 5 wt %.

44. The composition of any one of claims 41-43, wherein the carbohydrate is sucrose or trehalose.

45. The composition of any one of claims 41-44, wherein the carbohydrate preserves the thermostability of the composition.

46. The composition of any one of claims 40-45, wherein the composition is lyophilized.

47. A method of eliciting an antibody response in a subject, the method comprising:
administering an immunogenic composition of any one of claims 40-46 to the subject in an amount sufficient to elicit an antibody response.

48. The method of claim 47, wherein the subject is a mammal.

49. The method of claim 48, wherein the subject is a mouse.

50. The method of claim 48, wherein the subject is a human.

51. A method of making a multivalent immunogenic composition, the method comprising:
providing a host cell comprising (i) a modified genomic DNA of a first virus species comprising a nucleic acid encoding a matrix glycoprotein, wherein the modification creates a replication incompetent virus, comprises a deletion of a gene encoding a surface glycoprotein; and (ii) nucleic acids encoding surface glycoproteins from at least 2 virus species;
infecting the host cell with a helper virus of the first virus species to produce a population of particles comprising replication incompetent virions comprising the matrix protein of the first virus species and the surface glycoproteins from at least 2 virus species; and
isolating the population of particles.

52. The method of claim 51, wherein the helper virus is a Vesicular stomatitis virus (VSV).

53. A method of making a multivalent immunogenic composition, the method comprising:
transfecting a host cell with a nucleic acid encoding a matrix protein of one virus species and nucleic acids encoding surface glycoproteins of at least two virus species;
expressing viral proteins in the host cell to allow formation of particles comprising surface glycoproteins of at least two virus species;
purifying the particles;
formulating the multivalent immunogenic composition comprising the purified particles.

54. The method of claim 51, 52, or 53, wherein the host cell is selected from bacteria, mammalian cells, insect cells, yeast, and plant cells.

55. The method of claim 53, wherein the matrix protein is selected from a virus in one of the following families: paramyxovirus, filovirus, flavivirus, orthomyxovirus, arenavirus, retrovirus, coronavirus, matonavirus, togavirus, and alphavirus.

56. The method of claim 55, wherein the matrix protein is from a virus in the paramyxovirus family.

57. The method of claim 56, wherein the matrix protein is a Nipah virus matrix protein.

58. The method of claim 56, wherein the matrix protein is a Hendra virus matrix protein.

59. The method of any one of claims 53-58, wherein the genes for viral surface glycoproteins of interest are selected from fusion proteins (F), receptor binding glycoproteins (G), glycoprotein (GP), spike proteins (S), Neuraminidase (NA), amino-terminus glycoprotein (Gn), carboxyl-terminus glycoprotein (Gc), hemagglutinin (H), and P32 protein.

60. The method of claim 59, wherein the genes for viral surface glycoproteins of interest are selected from fusion proteins (F), receptor binding glycoproteins (G), and glycoprotein (GP).

61. The method of claim 59, wherein the genes for viral surface glycoproteins of interest are selected from spike proteins (S) and neuraminidase (NA).

62. The method of claim 59, wherein the genes for viral surface glycoproteins of interest are selected from amino-terminus glycoprotein (Gn), carboxyl-terminus glycoprotein (Gc), hemagglutinin (H), and P32 protein.

63. The method of claim 59, wherein the genes for viral surface glycoproteins of interest are multiple spike proteins.
